(19) United States
(12) Patent Application Publication
(10) Pub. No.: US 2022/0296722 A1
(43) Pub. Date: Sep. 22, 2022

(54) CONJUGATES AND CONJUGATES FOR USE IN PREVENTING OR TREATING OF BRAIN DAMAGE AND NEURODEGENERATIVE DISEASES

(71) Applicant: MEDICORTEX FINLAND OY, Turku (FI)

(72) Inventor: Adrian HAREL, Turku (FI)

(21) Appl. No.: 17/636,608

(22) PCT Filed: Aug. 17, 2020
(86) PCT No.: PCT/FI2020/050533

§ 371 Date: Feb. 18, 2022

(30) Foreign Application Priority Data
Aug. 30, 2019 (FI) 20195715

(51) Int. Cl. A61K 47/54; A61K 31/167; A61P 25/28; A61K 31/405; A61K 9/00

(52) U.S. Cl. CPC A61K 47/549; A61K 31/167; A61K 47/548; A61P 25/28; A61K 31/405; A61K 9/0053; A61K 9/0019; A61K 9/0073; A61K 9/0034; A61K 9/0031

(57) ABSTRACT

Disclosed are conjugates including one or more compounds selected from of (i) an anti-inflammatory compound, and/or (ii) an antioxidant compound, and/or (iii) and antibiotic compound and/or, (iv) a metal chelating compound and at least one of (v) a blood-brain barrier compound and (vi) solubility enhancer moiety. The compounds of group (i)-(v) and the solubility enhancer moiety (vi), when present, are covalently bound together with one or more linkers. The blood-brain barrier targeting compound is monosaccharide and the solubility enhancer moiety is a phosphate group. Also disclosed are pharmaceutical compositions including such conjugates and uses of these conjugates and compositions for treating and/or prevention of neuro-degenerative diseases and brain damage due to traumatic and/or acquired brain injury.

### FIELD OF THE INVENTION

The present invention relates to conjugates comprising molecules selected from an anti-inflammatory compound, an antioxidant compound, antibiotic compound, a metal chelating compound, and at least one blood-brain barrier targeting compound and a solubility enhancer. The present invention further provides pharmaceutical compositions including such conjugates and the use of these conjugates in treating or preventing brain damage due to traumatic and/or acquired brain injury and neurodegenerative diseases.

### BACKGROUND OF THE INVENTION

Traumatic brain injury (TBI) is a highly complex disorder that includes varying degrees of contusion, diffuse axonal injury, haemorrhage, and hypoxia. Collectively, these effects induce biochemical and metabolic changes that lead to progressive tissue damage and are associated with cell death. Both the early primary events and the delayed secondary alterations (secondary injuries) contribute to the resulting neurological deficits.

TBI is a major cause of morbidity and mortality, with an incidence of 235 per 100,000 people and a worldwide mortality of more than 1.5 million per year. The public health impact of TBI is expected to increase, since the most common cause of TBI is road traffic accidents. It is projected to become the fourth leading cause of disability by 2030. In addition, TBI is a major problem in combat areas/war zone, with a significant percentage of soldiers being reported to suffer from TBI.

Currently, TBI patients are treated with a combination of surgery, rehabilitation and pharmacological agents managing post-trauma conditions. However, although preclinical studies have suggested many promising pharmacological agents for the treatment of TBI, more than 30 phase III prospective clinical trials have failed to show significant effects for their primary endpoints. One major reason cited for these disappointing outcomes is that monotherapy approaches, that target single or limited mechanisms, are simply not adequate to address the complex and dynamic milieu of the injured brain. In recognition of the limitations of the monotherapy approach to treating TBI, increased attention is now being directed toward developing combination therapeutic strategies. Overall, the key criteria for a successful pre-clinical combination therapy is to (1) improve the therapeutic effects achieved via monotherapy through the synergistic interaction of two or more drugs administered in combination; and (2) to effectively lower the risk for adverse effects by using sub-threshold doses of the individual drugs in combination. Despite the high medical need, there are currently no pharmacological treatment options for TBI.

Neurodegeneration due to neurodegenerative brain disease and injury is a major health and economic concern. Current models predict at least more than a threefold rise in the total number of persons with Alzheimer's disease between 2000 and 2050. The well accepted concept of a basic cause for neurodegeneration is over-excitation by glutamate as well as impairments of mitochondria leading to death of nerve cells in the brain. Potential treatments have been designed and tested, for example, by inhibition of glutamate receptors. However, such approaches have not matured to effective treatments, probably because the causes for neurodegeneration are not well understood.

WO2016051024A1 discloses compounds for use in the treatment and prevention of brain damage. The compounds disclosed therein include a C10-25 alkenyl group as a blood-brain barrier penetrating head/tail. According to the document the compounds disclosed therein are able to cross the blood-brain barrier, bind free metals, prevent oxidative stress, inflammation, or infection, or provide an energy source for mitochondria.

US20060142181A1 discloses non-steroidal anti-inflammatory drug complexes for treating neurogenetic conditions. The complexes are metal chelates comprising the drug and a ligand operative in transport across the blood-brain barrier.

W02012016965A1 discloses compositions and methods for the treatment of traumatic brain injury. The compositions disclosed therein are conjugates of polymers and one or more anti-inflammatory agents.

However, there is still a need in the art to develop novel agents that effectively prevent and/or treat acquired and/or traumatic brain injury and neurodegenerative diseases. Thus, a need in the art exists to develop novel agents that effectively prevent and/or treat acquired and/or traumatic brain injury and neurodegenerative diseases.

### BRIEF DESCRIPTION OF THE INVENTION

An object of the present invention is thus to provide conjugates and compositions to overcome the above disadvantages. The objects of the invention are achieved by providing conjugates and pharmaceutical compositions, and the use of conjugates and pharmaceutical compositions that are characterized by what is stated in the independent claims. The preferred embodiments of the invention are disclosed in the dependent claims.

More exactly, the present invention provides said conjugates and pharmaceutical compositions for use in treating or preventing brain damage resulting from a brain injury and neurodegenerative diseases.

The invention is based on the surprising realization that compositions, conjugates and pharmaceutical compositions of the present invention can be used for treating or preventing brain damage resulting from a brain injury and neurodegenerative diseases. The conjugates, compositions and pharmaceutical compositions of the present invention have not been disclosed in the prior art as possessing any pharmacological activity per se.

### DETAILED DESCRIPTION OF THE PRESENT INVENTION

According to one embodiment the present invention concerns a conjugate comprising one or more compounds selected from (i) an anti-inflammatory compound; (ii) an antioxidant compound; (iii) an antibiotic compound; (iv) a metal chelating compound and at least one (v) a blood-brain barrier targeting compound or (vi) a solubility enhancer moiety. Naturally, the conjugate may comprise the blood-brain barrier targeting compound and the solubility enhancer moiety.

The solubility enhancer moiety and the compounds of group (i)-(v), when present, are covalently attached to each other via one or more linkers. The one or more linkers replace a hydrogen or a heteroatom anywhere in the compounds and the solubility enhancer moiety. The one or more linkers are formed from one or more moieties each independently selected from a group consisting of unsubstituted or substituted C1-C10 alkylene, unsubstituted or substituted C2-C10 alkenylene, unsubstituted or substituted C2-C10 alkynylene, unsubstituted or substituted arylene, amide, ester, ether, thioether, amine, phosphonate, urea, sulfonamide, ethyleneglycol, polyethylene glycol, and carbamate. According to the present invention the blood-brain barrier targeting compound of the conjugate is a monosaccharide, preferably glucose, and the solubility enhancer moiety is a phosphate group.

As defined herein a conjugate refers to a compound formed by the joining of two or more chemical compounds or molecules covalently. Accordingly, a covalent bond is formed between the linker and the compound or the molecule.

For example, if an antibiotic compound includes a carboxylic acid group (—COOH) it can be coupled to a linker compound including an amino function (NH₂—). In a chemical reaction a carboxamide bond (—CONH—) is formed. Accordingly, it is obvious for a skilled person that the compounds of group (i)-(v) of the conjugates of the present disclosure are not intact compounds but fragments thereof when they are tethered to the one or more linkers.

As defined herein an anti-inflammatory compound is a drug or a substance that reduces inflammation such as redness, swelling, and pain in the body. Anti-inflammatory agents block certain substances in the body that cause inflammation. Exemplary anti-inflammatory compounds suitable for the present disclosure are indomethacin, naproxen, acemetacin, acetyl salicylic acid, alclofenac, alminoprofen, azapropazone, benorylate, benoxaprofen, bucloxic acid, carprofen, choline magnesium trisalicylate, clidanac, clopinac, dapsone, diclofenac, diflunisal, droxicam, etodolac, fenoprofen, fenbufen, fenclofenec, fentiazac, floctafenine, flufenisal, flurbiprofen, (r)-flurbiprofen, (s)-flurbiprofen, furofenac, feprazone, flufenamic acid, fluprofen, ibufenac, ibuprofen, indoprofen, isoxepac, isoxicam, keto-profen, ketorolac, miroprofen, piroxicam, meloxicam, mefenamic, mefenamic acid, meclofenamic acid, meclofen, nabumetone, niflumic acid, oxaprozin, oxipinac, oxy-phenbutazone, phenylbutazone, podophyllotoxin derivatives, proglumetacin, piprofen, pirprofen, prapoprofen, salicylic acid, salicylate, sudoxicam, suprofen, sulindac, tenoxicam, tiaprofenic acid, tiopinac, tioxaprofen, tolfenamic acid, tolmetin, zidometacin, zomepirac, and 2-fluoro-a-methyl[1,1′-biphenyl]-4-acetic acid, 4-(nitrooxy)butyl ester, butylated hydroxyanisole, butylated hydroxytoluene, propyl gallate, tert-butyl hydroquinone (TBHQ), Gossypol, Tocopherols, and tocotrienols.

As defined herein an antioxidant is a compounds that inhibits oxidation. Oxidation is a chemical reaction that can produce free radicals, thereby leading to chain reactions that may damage the cells of organisms. Antioxidants terminate these chain reactions. Exemplary antioxidant compounds suitable for the present disclosure are resveratrol, chalcone (such as butein), flavanol (such as catechins and epicatechins), flavone (such as apigenin, baicalein, chrysin, diosmin, luteolin, scutellarein, tangeritin, and wogonin), flavonol (such as quercetin, kaempferol, myricetin, fisetin, isorhamnetin, pachypodol, and rhamnazin), hydroxycinnamic acids (such as caffeic acid, cichoric acid, chlorogenic acid, caftaric acid, coumaric acid, coutaric acid, diferulic acids, fertaric acid, and ferulic acid) and stilbenoids (such as piceatannol).

As defined herein an antibiotic compound is a type of antimicrobial substance active against bacteria.

As defined herein polyketides are secondary metabolites which either contain alternating carbonyl groups and methylene groups (—CO—CH₂—) or are derived from precursors which contain such alternating groups. Exemplary polyketide antibiotic compounds are tetracycline, doxycycline, minocycline, chlortetracycline, oxytetracycline, demeclocycline, lymecycline, meclocycline, methacycline, rolitetracycline and tigecycline.

As defined herein metal chelating compounds refers to an organic moiety that binds with and removes free metal ions from solution. Exemplary metal chelating compound suitable for the present disclosure are citric acid, ascorbic acid, ethylene diamine tetra acetate (EDTA), desferrioxamine, and d-penicillamine, natural chelating agents such as lactoferrin, inositol hexaphosphate (IP6), ferulic acid, curcumin, ellagic acid, hydroxytyrosol, anthocyanidin, 4-{[(1,2-dimethyl-1,2-dihydropyridin-4-yl)methyl](ethyl)amino}butanoic acid, dihydroxybenzoic acid, catechol, benzoic acid optionally substituted with 1-5 OR2, wherein R2 is independently at each occurrence selected from a group consisting of H, C1-C10-alkyl, C2-C6-alkenyl, C2-C6-alkynyl, C3-C8-cycloalkyl, heterocyclyl, heteroaryl, aryl, and —C(O)R3, each of which may be unsubstituted or substituted, where-in R3 has the same meaning as R2; an unsubstituted or substituted mono- or bicyclic arylene or heteroarylene, substituted with 0-5 R1 that is independently at each occurrence selected from a group consisting of OH and a functional group capable of metal chelation, and a catechol moiety of said R1 and said unsubstituted or substituted mono- or bicyclic arylene or heteroarylene that together with or without a hydroxyl of a sugar moiety forms a catechol moiety.

As defined herein a blood-brain barrier (BBB) targeting compound is a compound that able to transfer drugs across BBB. The BBB-targeting compounds of the present disclosure are monosaccharides.

Prior therapeutic approaches for the treatment of TBI are typically based on single agent treatment paradigms that target a single factor thought to mediate secondary injury. The present invention is based on the realization that the complexity and diversity of the secondary injury mechanisms warrants being treated by targeting multiple delayed injury factors. This may be achieved by combining agents that have complementary effects or by using multi-potential drugs that modulate multiple injury mechanisms. Thus, the present invention is based on a unique combination of activities targeting several pathways mediating secondary injury.

More exactly, the present invention is based on a conjugate that incorporates at least two biochemical functional activities, such as free metal ion chelation, anti-oxidant, free radical scavengers, and anti-inflammatory like activity, linked via a linker to a penetrating head (for access through the blood-brain barrier (BBB)) or to a solubility enhancer moiety. These conjugates possess properties for preventing secondary brain deterioration in TBI patients, and/or for treatment of neurodegenerative diseases. These conjugates are drug candidates for the treatment of acquired brain injury (ABI), including traumatic brain injury (TBI), and non-traumatic brain injury. TBI is caused by something that comes from outside of the body such as blow, bump, or jolt. Non-traumatic brain injury is caused by conditions like ischemic stroke, haemorrhagic stroke, aneurysm, seizure disorders, brain tumour, poisoning, substance abuse, opioid overdose, meningitis, encephalitis, hydrocephalus, vasculitis, or hematoma. In addition, these conjugates are drug candidates for the treatment of other diseases including brain injuries and neurodegenerative diseases. TBI, ABI, stroke, and related diseases, are characterized by a chain of primary and secondary biochemical events leading to damage at the cellular level and possible long-term effect. The object of the present invention is to simultaneously and consequently address several biochemical pathways by incorporating multiple biochemical functionalities into a single molecule and coupling with a targeting moiety that will direct brain penetration. An intervention that simultaneously targets multiple factors which contribute to the progress of neuro-degradation, could also be more effective in halting secondary TBI, as well as play a role in neuroprotection. The unique, multi-functional approach aiming both at decreasing circulating toxic metal ion levels in the brain as well as minimizing oxidation by these free radicals, has not been described before. This synergistic approach is expected to prevent the cascade of events leading to brain degeneration.

In one embodiment, a plurality of said compounds are covalently linked to each other, while the others optionally may be added as combination treatment, in the same pharmaceutical composition, or in a separate composition intended for concomitant or sequential administration. In another embodiment, the plurality of compounds is covalently linked by chemical spacers (linkers) so as to form a conjugate combining all therapeutic functionalities into a single agent. In another embodiment, one compound in the combination may possess multiple therapeutic functionalities (e.g., an antioxidant and an anti-inflammatory). Each possibility represents a separate embodiment of the present invention.

In one embodiment, the present invention relates to hydroxybenzoic acid and derivatives thereof, conjugated by a linker (L) to a sugar moiety and optionally to a metal chelator.

In some embodiments, the compounds of the invention are composed of a dihydroxybenzoic acid moiety, with or without a catechol group able to chelate metals, conjugated by a linker (L) to a sugar subunit, preferably glucose linked by its C-6 position. It is well known that glucose has high transport capacity of the carrier-mediated transporters present at the blood-brain barrier (BBB) being therefore an attractive transporter for prodrug delivery. In accordance with one embodiment, once the compound is administered into living cells, the sugar moiety is cleaved from the molecule to generate an active drug. Thus, the compounds of the invention are particularly advantageous as prodrugs using the sugar subunit as a targeting moiety to the brain, thereby enhancing the delivery of the active moiety to its site of action. Targeted delivery may reduce the dosage required for treatment effect, and the associated toxicities and/or side effects may be reduced accordingly. In addition to functioning as a BBB-targeting moiety, the sugar group also serves as energy source/supply.

The compositions of the invention are particularly advantageous as the combinations described herein may allow for the use of reduced dosages of each component, thereby reducing toxicities and/or side effects associated with each of the components of the combination. Furthermore, the combinations may exhibit a synergistic/complementary/additive effect in the treatment and/or prevention of acquired and/or traumatic brain injury and neurodegenerative diseases, thus further reducing the dose (and associated toxicities) associated with each of the individual components.

Advantageously, compounds having multiple therapeutic functionalities may be incorporated, thus reducing the number of individual compounds used for the combinations of the invention. For example, a compound may possess anti-inflammatory and antioxidant activity; or an antibiotic and antioxidant activity, and the like.

Thus, according to one aspect, the present invention provides a composition comprising a combination of a plurality of compounds selected from: (i) an anti-inflammatory compound or a fragment thereof; (ii) an anti-oxidant compound or a fragment thereof; (iii) an antibiotic compound or a fragment thereof; (iv) a metal chelating compound or a fragment thereof; and (v) a blood-brain barrier targeting compound or a fragment thereof, wherein at least two of said compounds, or fragments thereof, are covalently attached to each other via at least one linker to form a conjugate.

According to another aspect, the present invention provides a conjugate comprising a plurality of compounds selected from: (i) an anti-inflammatory compound or a fragment thereof; (ii) an anti-oxidant or free radical scavenger compound or a fragment thereof; (iii) a polyketide antibiotic compound or a fragment thereof; (iv) a metal chelating compound or a fragment thereof; and (v) a blood-brain barrier targeting compound or a fragment thereof, each covalently attached to each other via at least one linker to form a conjugate.

In some embodiments, at least one compound in said composition comprises at least two therapeutic functionalities selected from antioxidant, antibiotic, anti-inflammatory, metal chelation and blood-brain barrier targeting.

In one currently preferred embodiment, the anti-inflammatory compound is indomethacin or naproxen.

In one currently preferred embodiment, the antioxidant compound is resveratrol.

In one currently preferred embodiment, the antibiotic compound is a polyketide antibiotic compound tetracycline.

In one currently preferred embodiment, the metal chelating compound is derived from 4-{[(1,2-dimethyl-1,2-dihydropyridin-4-yl)methyl](ethy)amino}butanoic acid.

In one currently preferred embodiment, the blood-brain-barrier targeting compound is glucose.

According to some embodiments, at least one of the aforementioned compounds (i), (ii), (iii), (iv) and (v) is further attached, directly or through a linker, to a further solubility enhancer moiety, which may in some embodiments comprises a phosphate group.

In some embodiments, the compositions of the present invention penetrate the blood-brain barrier (BBB), for administration directly into the central nervous system (CNS).

According to some embodiments, the composition of the invention comprises a conjugate represented by the structure of Formula (I):

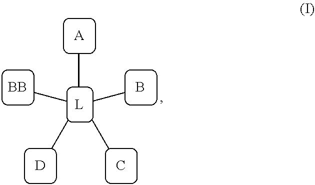

wherein:
A is an anti-inflammatory compound;
B is an antioxidant compound;
C is an antibiotic compound;
D is a metal chelating compound;
BB is a a blood-brain-barrier targeting compound
L comprises at least one linker;
wherein each of A, B, C, D, and BB is optional.
wherein at least one of A, B, C, D and BB is optionally further covalently linked, directly or through a linker to a solubility enhancer moiety;
or salts, hydrates, solvates, polymorphs, optical isomers, geometrical isomers, enantiomers, diastereomers and mixtures thereof.

However, at least one of the compound A, B, C or D must be present, and either the blood-brain barrier targeting compound BB or the solubility enhancer moiety must be present. In the conjugate, the solubility enhancer and the BB compound is phosphate and monosaccharide, respectively.

The term “linker” as used herein and hereafter is to be understood as a chemical moiety (radical), linking a plurality of said A, B, C, D, and BB compounds and the solubility enhancer moiety forming a conjugate. Hydrogens removed from the carbon chain forming the di- to multivalent radicals can all be hydrogens of terminal carbons, hydrogens of each of two terminal carbons or hydrogens of intermediate carbon atoms, or combinations thereof. Examples of linkers include, but are not limited to, a bond, an unsubstituted or substituted, branched or linear, C1-C10-alkylene, C2-C10 alkenylene, C2-C10 alkynylene, di- to multivalent radicals derived from, unsubstituted or substituted, aryl, ethyleneglycol, polyethylene glycol, heteroaryl, cycle, heterocycle, amide, ester, ether, thioether, amine, phosphonate, urea, sulfonamide or carbamate, or any combination thereof. More specifically, examples of linkers include, but are not limited to, methanediylidene, methylene, methylidyne, cyclohexylene, and phenylene.

In one embodiment, the linker is a C1-C10 alkylene, C2-C10 alkenylene, C2-C10 alkynylene, aryl, heteroaryl, cycle, or heterocycle, bound to at least one functional group, each independently selected from an amide, ester, ether, thioether, amine, phosphonate, urea, sulfonamide or carbamate, wherein the anti-inflammatory compound, the anti-oxidant compound, the polyketide antibiotic compound, the metal chelating compound and/or the blood-brain-barrier targeting compound, or fragments thereof, are bound with said functional group(s) to the rest of the conjugate. Each possibility represents a separate embodiment of the present invention.

It is to be understood that each linker can be independently attached to another linker. In one embodiment, said A, B, C, D, and/or BB compounds, and/or fragments thereof, are conjugated together with one linker, e.g. L. In another embodiment, said A, B, C, D, and/or BB compounds, and/or fragments thereof, are linked together with two linkers, wherein the two linkers are attached to each other, i.e. linker L consist of linkers L1 and L2. In another embodiment, said A, B, C, D, and/or BB compounds, and/or fragments thereof, are conjugated together with three linkers, wherein the one or two of said three linkers are attached to one or two of said linkers, i.e. linker L consist of linkers L1, L2, and L3.

According to some embodiments, the conjugate of Formula (I) is represented by the structure of Formula (II):

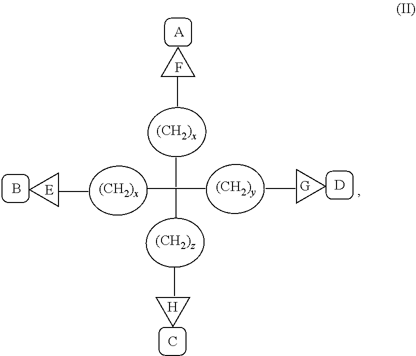

wherein:
A, B, C, and D are as defined hereinabove;
x, y, z, and n are each independently an integer 0-10; and
E, F, G, and H are each independently absent or a functional group selected from the group consisting of amide, ester, ether, thioether, amine, phosphonate, urea, sulfonamide and carbamate; and
at least one of A, B, C, and D is further covalently linked, through a linker to a solubility enhancer moiety or a blood-brain barrier targeting compound (BB).
or salts, hydrates, solvates, polymorphs, optical isomers, geometrical isomers, enantiomers, diastereomers and mixtures thereof.

Exemplary linker types are shown below, wherein each • represents A, B, C,D, BB or solubility enhancer moiety as defined above. The solid line therebetween is a linker.

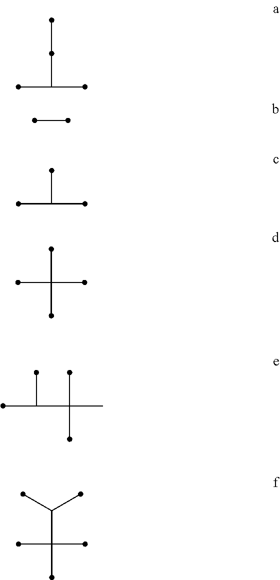

The example a includes two divalent linkers, while the examples b-f include 2-,3-,4-,5- and 6-valent linkers, respectively.

According to a currently preferred embodiment, the linker comprises a C1-C10 alkylene-amide or a C1-C10 alkylene-amine moiety.

In one currently preferred embodiment, the anti-inflammatory compound is indomethacin or naproxen or a fragment thereof. In another currently preferred embodiment, the antioxidant compound is resveratrol or a fragment thereof. In another currently preferred embodiment, the polyketide antibiotic compound is tetracycline or a fragment thereof. In another currently preferred embodiment, the metal chelating compound is derived from 4-{[(1,2-dimethyl-1,2-dihydropyridin-4-yl)methyl](ethyl)-amino}butanoic acid, which can be bound to the conjugate through any available atoms or through, e.g., an amide or ester bond. In another currently preferred embodiment, the blood-brain-barrier targeting compound is glucose or a fragment thereof. In another currently preferred embodiment, the solubility enhancer moiety is a phosphate group.

According to some embodiments, the conjugate of the present disclosure is represented by the structure of Formula (III):

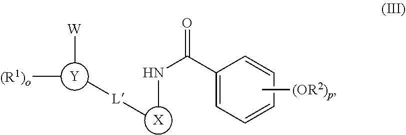

wherein
   both X and Y together with L′ forms the linker L, and X and Y each are independently an unsubstituted or substituted mono- or bicyclic arylene or heteroarylene;

W is a monosaccharide selected from the group consisting of a pentose saccharide or a fragment thereof, a hexose saccharide or a fragment thereof, anti-inflammatory compound or a fragment thereof, anti-oxidant compound or a fragment thereof, or a polyketide antibiotic compound or a fragment thereof, as described hereinabove;

L′ is a linker selected from a group consisting of bond, an unsubstituted or substituted, linear or branched, C1-C10-alkylene, C2-C10-alkenylene and C2-C10-alkynylene, each of which is optionally substituted with a group selected from the group consisting of —O—, —S—, —NH—, —C(═O)—, —C(═O)—O—, —C(═O)—NH—, —NH—C(═O)—NH—, NH—C(═O)—O—, —S(═O)—, —S(═O)—O—, PO(═O)O—, and any combination thereof;

R¹ is independently at each occurrence selected from a group consisting of OH and a functional group capable of metal chelation, or (R¹)ₙ—B together form a functional group capable of metal chelation;

R² is independently at each occurrence selected from a group consisting of H, C1-C10-alkyl, C2-C6-alkenyl, C2-C6-alkynyl, C3-C8-cycloalkyl, heterocyclyl, heteroaryl, aryl, and —C(O)R³, each of which may be unsubstituted or substituted, wherein R³ has the same meaning as R²;
p is 1, 2, 3, 4 or 5; and
o is 0, 1, 2, 3, 4 or 5;
or salts, hydrates, solvates, polymorphs, optical isomers, geometrical isomers, enantiomers, diastereomers, and mixtures thereof.

According to some embodiments, the conjugate of Formula (I) is represented by the structure of Formula (Ill), wherein
X and Y are each independently an unsubstituted or substituted mono- or bicyclic arylene or heteroarylene;
W is selected from the group consisting of a pentose saccharide or a fragment thereof, a hexose saccharide or a fragment thereof, anti-inflammatory compound or a fragment thereof, anti-oxidant compound or a fragment thereof, or a polyketide antibiotic compound or a fragment thereof, as described hereinabove;
L′ is a linker selected from a group consisting of a bond, an unsubstituted or substituted, linear or branched, C1-C10-alkylene, C2-C10-alkenylene and C2-C10-alkynylene, each of which is optionally substituted with a group selected from the group consisting of —O—, —S—, —NH—, —C(═O)—, —C(═O)—O—, —C(═O)—NH—, —NH—C(═O)—NH—, NH—C(═O)—O—, —S(═O)—, —S(═O)—O—, PO(═O)O—, and any combination thereof;
R¹ is independently at each occurrence selected from a group consisting of OH and a functional group capable of metal chelation, or (R¹)ₙ—B together form a functional group capable of metal chelation;
R² is independently at each occurrence selected from a group consisting of H, C1-C10-alkyl, C2-C6-alkenyl, C2-C6-alkynyl, C3-C8-cycloalkyl, heterocyclyl, heteroaryl, aryl, and -C(O)R³, each of which may be unsubstituted or substituted, wherein R³ has the same meaning as R²;
p is 1, 2, 3, 4 or 5; and
o is 0, 1, 2, 3, 4 or 5;
or salts, hydrates, solvates, polymorphs, optical isomers, geometrical isomers, enantiomers, diastereomers, and mixtures thereof.

In one embodiment, ring X is a phenylene, i.e., a phenyl moiety bound to the linker L′ and the amide moiety.

In one embodiment, ring Y is a phenylene, i.e., a phenyl moiety bound to the linker L′ and to a sugar moiety W, and optionally substituted with one or more R¹ substituents. In one embodiment, o is at least 1 and R¹ is any of the substituents described above. In one embodiment, R¹ is OH. In another embodiment R¹ is OH and is located ortho to the sugar moiety W. In accordance with this embodiment, R¹-B together with a hydroxyl of the sugar moiety forms a catechol moiety:

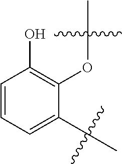 [SMILES: *Oc1c(*)cccc1O]

In one embodiment, o is 2 and each R¹ is OH. In accordance with this embodiment, R¹-B forms a different catechol moiety:

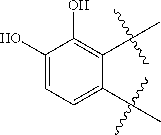 [SMILES: *c1ccc(O)c(O)c1*]

In another embodiment, o is 0 and R¹ does not exist. In accordance with this embodiment, the compound of the invention does not contain a catechol moiety between the amide moiety and the sugar moiety. Each possibility represents a separate embodiment of the present invention.

The term “functional group capable of metal chelation” as used herein, refers to an organic moiety that binds with and removes free metal ions from solution. A currently preferred functional group capable of metal chelation is a catechol moiety. In accordance with this embodiment, the compounds of formula (III) may contain an optional catechol moiety as part of the molecular structure. Examples of such catechol structures include, but are not limited to, compounds of formula (III), in which R¹-B with or without the hydroxyl of the sugar moiety forms a catechol moiety, as depicted in compounds (3-5) herein.

In one preferred embodiment, W is a moiety derived from a sugar (saccharide) which may be a pentose or a hexose. In addition to functioning as a BBB-targeting moiety, the sugar group also serves as energy source/supply. The sugar may be in its linear (open chain form) or in ring form (pyranose or furanose), or a combination thereof. In one embodiment, the sugar is a monosaccharide. Non-limiting examples of sugars that are suitable for the compositions of the present invention are allose, altrose, glucose, mannose, gulose, idose, galactose, talose, psicose, fructose, sorbose, tagatose, ribose, arabinose, xylose, lyxose, ribulose, and xylulose. In other embodiments, the sugar may be a deoxy sugar, i.e., a sugar that has one or more of the hydroxyl group replaced with a hydrogen atom (including dideoxy sugars, trideoxy sugars, and the like). Examples include deoxyribose, fucose or rhamnose. The sugar may also be an amino sugar, pseudo-sugar, or thio-sugar, each possibility represents a separate embodiment of the present invention.

The sugar may be bound through any available oxygen atoms to the rest of the molecule. The sugar may also be linked through any available nitrogen, sulfur or carbon atom. Thus, the sugar can be linked by an O—(O-glycoside), N—(glycosylamine), S—(thioglycoside), or C—(C-glycoside) glycosidic bond. In another embodiment, the sugar may be in the form of an acetal or thioacetal.

For example, when the sugar is a pentose, it may be bonded through a hydroxyl/amino/thio/carbon at C-1, C-2, C-3, C-4 or C-5. When the sugar is a hexose, it may be bonded through a hydroxyl/amino/thio/carbon at C-1, C-2, C-3, C-4, C-5 or C-6. In one preferred embodiment, W is glucose bonded through its C-6 hydroxyl, which is represented by the structure:

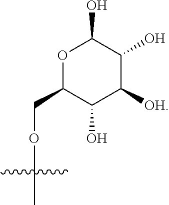 [SMILES: *OC[C@H]1O[C@@H](O)[C@H](O)[C@@H](O)[C@@H]1O]

The hydroxyls, when present, may be present in the equatorial or axial position of the sugar ring (beta or alpha positions). Each possibility represents a separate embodiment of the present invention.

The moiety

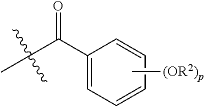

is preferably derived from a hydroxybenzoic acid or its analogs and derivatives. In one embodiment, R² is H, thereby defining a hydroxybenzoic acid derivative. In one embodiment, p is 1. In another embodiment, p is 2, and the above moiety is derived from dihydroxybenzoic acid (DHB). Dihydroxybenzoic acids are a type of phenolic acids. There are six main compounds, having all the same molecular formula C₇H₆O₄. Those are: 1) 2,3-dihydroxybenzoic acid (2-pyrocatechuic acid or hypogallic acid); 2) 2,4-dihydroxybenzoic acid (β-resorcylic acid; 3) 2,5-dihydroxybenzoic acid (gentisic acid); 4) 2,6-dihydroxybenzoic acid (γ-resorcylic acid); 5) 3,4-dihydroxybenzoic acid (protocatechuic acid); and 6) 3,5-dihydroxybenzoic acid (α-resorcylic acid). 2,3-DHB and 3,4-DHB contain a catechol group, which upon deprotonation binds iron centers. All DHBs contain a carboxylic acid group by which the ring attaches to various scaffolds via, e.g., amide linkages.

In one embodiment of the present invention, the moiety

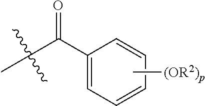

is derived from any of the aforementioned DHBs, with each possibility representing a separate embodiment of the present invention. In other embodiments, the aforementioned moiety may be derived from hydroxybenzoic acid derivatives, such as compounds wherein R² is a C1-C10 alkyl, e.g., methoxyphenylbenzamide or ethoxyphenylbenzamide derivatives. Each possibility represents a separate embodiment of the present invention.

The linker L′ varies and is generally an alkylene, alkenylene or alkynylene which may be substituted at any position with a functional group selected from —O—, —S—, —NH—, —C(═O)—, —C(═O)—O—, —C(═O)—NH—, —NH—C(═O)—NH—, NH—C(═O)—O—, —S(═O)—, —S(═O)—O—, PO(═O)O—. One linker is ethylene glycol, propylene glycol and the like. In one embodiment, L′ is —Y—(CH₂)₂-Het wherein z is 1 to 10 and Het is independently at each occurrence O, S or NH.

In one currently preferred embodiment, the composition of the invention is represented by the structure of formula (IV):

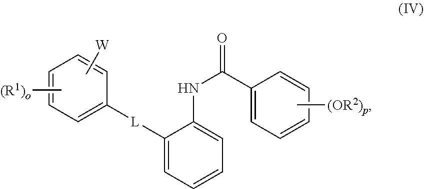

wherein W, R¹, R², L, o, and p, are as defined as for formula III. In one embodiment, groups W and L are positioned ortho (adjacent) to each other.

Specific examples of the conjugates of formula I, II, Ill, and IV include, but are not limited to, either one of the following structures:

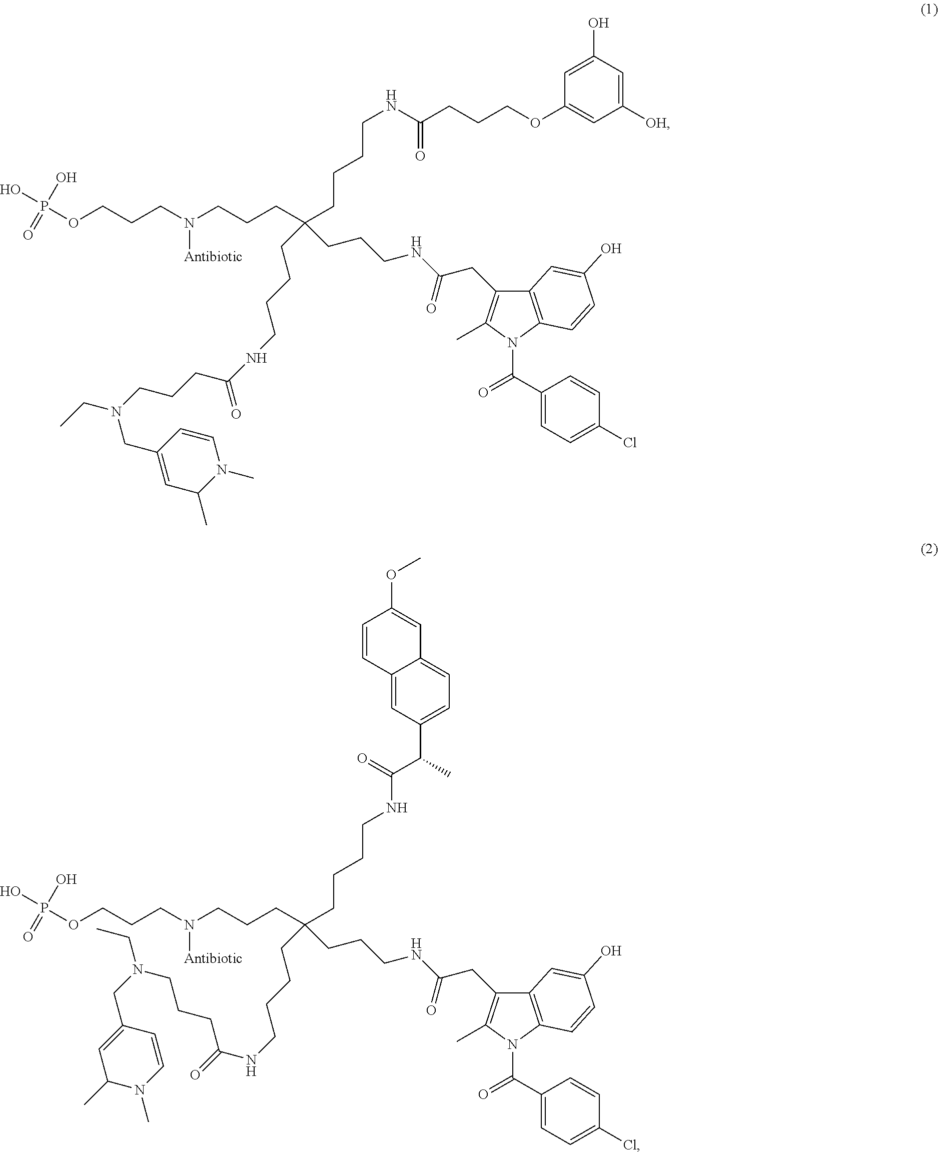

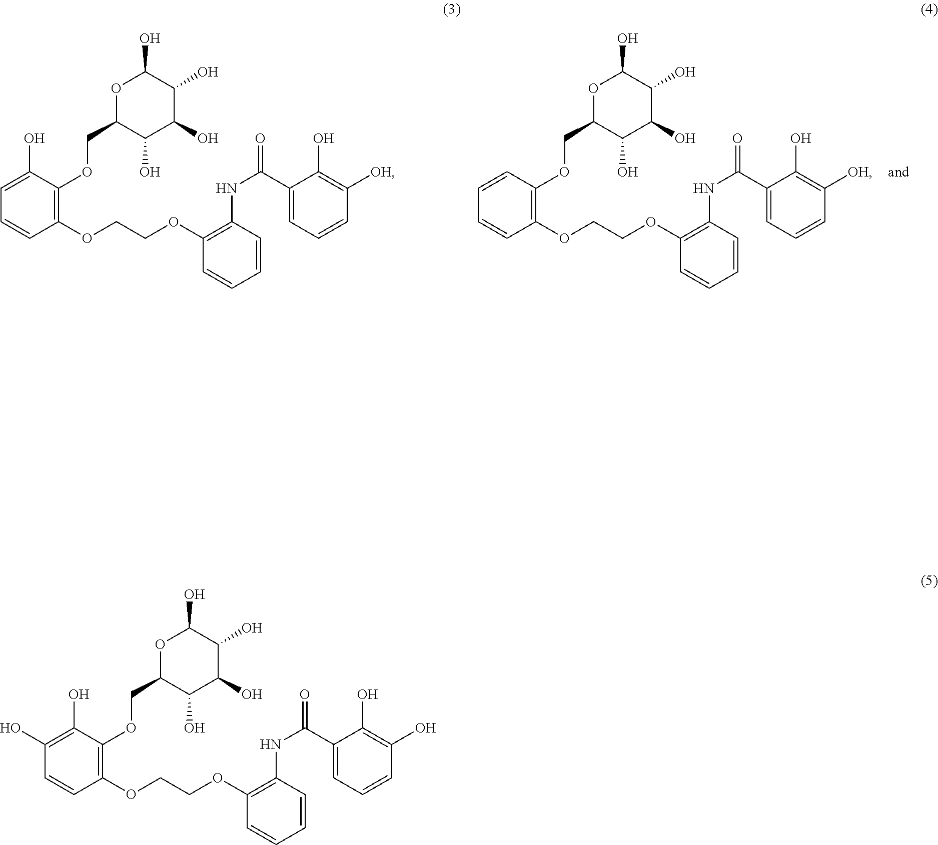 [SMILES: O=C(Nc1ccccc1OCCOc1cccc(O)c1OC[C@H]1O[C@@H](O)[C@H](O)[C@@H](O)[C@@H]1O)c1cccc(O)c1O ; O=C(Nc1ccccc1OCCOc1ccccc1OC[C@H]1O[C@@H](O)[C@H](O)[C@@H](O)[C@@H]1O)c1cccc(O)c1O ; "and" ; O=C(Nc1ccccc1OCCOc1ccc(O)c(O)c1OC[C@H]1O[C@@H](O)[C@H](O)[C@@H](O)[C@@H]1O)c1cccc(O)c1O]

According to another aspect, the present invention provides a pharmaceutical composition comprising as an active ingredient the composition as described herein, and a pharmaceutically acceptable carrier and/or excipient.

According to some embodiments, the pharmaceutical composition is in the form of a capsule, tablet, granule, powder, solution, polymer or suspension and designed for administration by an oral sublingual, buccal, parenteral, intravenous, transdermal, inhalation, intranasal, vaginal, intramuscular or rectal mode.

The compositions of the invention are useful in the treatment and prevention of brain damage resulting from traumatic and/or acquired brain injury (TBI and ABI, respectively), especially secondary brain injury due to TBI associated with, e.g., warfare, automobile accidents, sports injuries, violent crimes, household accidents, child abuse, concussion, gun-shot wounds, etc. In addition to brain injury, the compositions of the invention are also useful in treating and/or preventing neurodegenerative diseases such as Alzheimer's Disease, Multiple System Atrophy (MSA), Amyotrophic Lateral Sclerosis (ALS), and Parkinsonism (i.e., Parkinson's syndrome, atypical Parkinson's, or secondary Parkinson's, including Parkinson's Disease), among others. Each possibility represents a separate embodiment of the present invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

The term “C1-C10 alkyl” as used herein refers to any saturated aliphatic hydrocarbon of 1 to 10 carbon atoms. Examples of alkyl groups include, but are not limited to, methyl, ethyl, propyl, isopropyl, n-butyl, sec-butyl, t-butyl and the like.

The term “C2-C6 alkenyl” as used herein refers to an aliphatic hydrocarbon group containing at least one carbon-carbon double bond including straight-chain and branched-chain groups. Exemplary alkenyl groups include, but are not limited to, ethenyl, propenyl, n-butenyl, i-butenyl, 3-methylbut-2-enyl, n-pentenyl and the like.

The term “C2-C6 alkynyl” as used herein refers to an aliphatic hydrocarbon group containing at least one carbon-carbon triple bond including straight-chain and branched-chain groups. Exemplary alkynyl groups include, but are not limited to, ethynyl, propynyl, n-butynyl, 2-butynyl, 3-methylbutynyl, n-pentynyl, and the like.

The term “C3-C8 cycloalkyl” as used herein refers to cycloalkyl of 3 to 8 carbon atoms which includes monocyclic or polycyclic groups. Non-limiting examples of cycloalkyl groups are cyclopropyl, cyclobutyl, cyclopentyl, cyclohexyl or cycloheptyl.

The term “aryl” used herein alone or as part of another group denotes an aromatic ring system containing from 6-14 ring carbon atoms. The aryl ring can be a monocyclic, bicyclic, tricyclic and the like. Non-limiting examples of aryl groups are phenyl, naphthyl including 1-naphthyl and 2-naphthyl, and the like.

The term “heterocyclic ring” or “heterocyclyl” used herein alone or as part of another group refers to a five-membered to eight-membered rings that have 1 to 4 heteroatoms, such as oxygen, sulfur and/or nitrogen, in particular nitrogen, either alone or in conjunction with sulfur or oxygen ring atoms. These five-membered to eight-membered rings can be saturated, fully unsaturated or partially unsaturated, with fully saturated rings being preferred. Preferred heterocyclic rings include, but are not limited to, piperidinyl, pyrrolidinyl pyrrolinyl, pyrazolinyl, pyrazolidinyl, morpholinyl, thiomorpholinyl, pyranyl, thiopyranyl, piperazinyl, indolinyl, dihydrofuranyl, tetrahydrofuranyl, dihydrothiophenyl, tetrahydrothiophenyl, dihydropyranyl, tetrahydropyranyl, dihydrothiazolyl, and the like.

The term “heteroaryl” used herein denotes a heteroaromatic system containing at least one heteroatom ring atom selected from nitrogen, sulfur and oxygen. The heteroaryl generally contains 5 or more ring atoms. The heteroaryl group can be monocyclic, bicyclic, tricyclic and the like. Also included in this expression are the benzoheterocyclic rings. If nitrogen is a ring atom, the present invention also contemplates the N-oxides of the nitrogen containing heteroaryls. Non-limiting examples of heteroaryls include thienyl, benzothienyl, 1-naphthothienyl, thianthrenyl, furyl, benzofuryl, pyrrolyl, imidazolyl, pyrazolyl, pyridyl, pyrazinyl, pyrimidinyl, pyridazinyl, indolyl, isoindolyl, indazolyl, purinyl, isoquinolyl, quinolyl, naphthyridinyl, quinoxalinyl, quinazolinyl, cinnolinyl, pteridinyl, carbolinyl, thiazolyl, oxazolyl, isothiazolyl, isoxazolyl and the like. Each possibility represents as separate embodiment of the present invention.

The term “C1-C10 alkylene” used herein alone or as part of another group denotes a bivalent radicals of 1 to 10 carbons, which is bonded at two positions connecting together two separate additional groups (e.g., CH₂). Said alkylene can be linear or branched. Examples of saturated alkylene groups include, but are not limited to, —(CH₂)—, —(CH₂)₂—, —(CH₂)₃—, —(CH₂)₄—, etc.

The term “C2-C10 alkenylene” denotes a bivalent radical of 2 to 10 carbons which contains at least one double bond, which is bonded at two positions connecting together two separate additional groups. Non-limiting example of alkenylene include, but is not limited to, —CH═CH—.

The term “C2-C10 alkynylene” denotes a bivalent radicals of 2 to 10 carbons containing at least one triple bond, which is bonded at two positions connecting together two separate additional groups. Non-limiting example of alkenylene include, but is not limited to —C≡C—.

The terms “arylene” and “phenylene” are interchangeable and denote a bivalent radicals of aryl, which is bonded at two positions connecting together two separate additional groups.

The term “heteroarylene” denotes a bivalent radicals of heteroaryl, which is bonded at two positions connecting together two separate additional groups.

Any of the moieties described herein may be unsubstituted, or substituted with one or more substituents selected from the group consisting of halogen, hydroxy, alkoxy, aryloxy, alkylaryloxy, heteroaryloxy, oxo, cycloalkyl, phenyl, heteroaryl, heterocyclyl, naphthyl, amino, alkylamino, arylamino, heteroarylamino, dialkylamino, diarylamino, alkylarylamino, alkylheteroarylamino, arylheteroarylamino, acyl, acyloxy, nitro, carboxy, carbamoyl, carboxamide, cyano, sulfonyl, sulfonylamino, sulfinyl, sulfinylamino, thiol, C₁ to C₁₀ alkylthio, arylthio, or C₁ to C₁₀ alkylsulfonyl groups. Any substituent can be unsubstituted or further substituted with any one of these aforementioned substituents. Each possibility represents as separate embodiment of the present invention.

As used herein, the terms “OH protecting group” or “hydroxy protecting group” are interchangeable and refer to a readily cleavable groups bonded to hydroxyl groups. The nature of the hydroxy-protecting groups is not critical so long as the derivatized hydroxyl group is stable. An example of a hydroxy protecting group is a benzyl group (Bn), which is cleavable with a hydrogenation reaction (e.g., H₂/Pd, C). Another example of a hydroxy protecting group is an alkyl (e.g., C₁-C₄ alkyl), which is removed by acid or Lewis acids such as BBr₃, which specifically deprotect methyl ether groups. Another example of a hydroxy-protecting group is a cyclic ether (e.g., tetrahydropyranyl (THP), which is removed under acidic conditions). Another example of a hydroxy-protecting group is an acyclic ether (e.g., β-methoxyethoxymethyl ether (MEM), monomethyl ether (MOM)) which is removed under acidic conditions. Another example of a hydroxy protecting group is an acyl group (COR wherein R═alkyl, aryl, etc.). Another example of a hydroxy protecting group is a silyl group, which can be substituted with alkyl (trialkylsilyl), with an aryl (triarylsilyl) or a combination thereof (e.g., dialkylphenylsilyl). A preferred example of a silyl protecting group is trimethylsilyl (TMS) or di-t-butyldimethyl silyl (TBDMS). Other examples of hydroxy protecting groups include, for example, —CO—(C₁-C₆ alkyl), —SO₂—(C₁-C₆ alkyl), —SO₂-aryl,—CO-Ar in which Ar is an aryl group as defined above, and —CO—(C₁-C₆ alkyl)Ar (e.g., a carboxybenzyl (Bz) group). Other examples of hydroxy-protecting groups are described by C. B. Reese and E. Haslam, “Protective Groups in Organic Chemistry,” J. G. W. McOmie, Ed., Plenum Press, New York, N.Y., 1973, Chapters 3 and 4, respectively, and T. W. Greene and P. G. M. Wuts, “Protective Groups in Organic Synthesis,” 2^{nd} ed., John Wiley and Sons, New York, N.Y., 1991, Chapters 2 and 3, each of which is incorporated herein by reference.

All stereoisomers, optical and geometrical isomers of the compounds of the instant invention are contemplated, either in admixture or in pure or substantially pure form. The compounds of the present invention can have asymmetric centres at any of the atoms. Consequently, the compounds can exist in enantiomeric or diastereomeric forms or in mixtures thereof. The present invention contemplates the use of any racemates (i.e., mixtures containing equal amounts of each enantiomers), enantiomerically enriched mixtures (i.e., mixtures enriched for one enantiomer), pure enantiomers or diastereomers, or any mixtures thereof. The chiral centres can be designated as R or S or R,S or d,D, I,L or d,l, D,L. In addition, several of the compounds of the invention contain one or more double bonds. The present invention intends to encompass all structural and geometrical isomers including cis, trans, E and Z isomers, independently at each occurrence.

One or more of the compounds of the invention, may be present as a salt. The term “salt” encompasses both basic and acid addition salts, including but not limited to phosphate, dihydrogen phosphate, hydrogen phosphate and phosphonate salts, and include salts formed with organic and inorganic anions and cations. Furthermore, the term includes salts that form by standard acid-base reactions of basic groups and organic or inorganic acids. Such acids include hydrochloric, hydrofluoric, hydrobromic, trifluoroacetic, sulfuric, phosphoric, acetic, succinic, citric, lactic, maleic, fumaric, cholic, pamoic, mucic, D-camphoric, phthalic, tartaric, salicyclic, methanesulfonic, benzenesulfonic, p-toluenesulfonic, sorbic, picric, benzoic, cinnamic, and like acids. Additional salts of the compositions described herein may be prepared by reacting the parent molecule with a suitable base, e.g., NaOH or KOH to yield the corresponding alkali metal salts, e.g., the sodium or potassium salts. Additional basic addition salts include ammonium salts (NH₄⁺), substituted ammonium salts, Li, Ca, Mg, salts, and the like.

The present invention also includes solvates of the compounds of the present invention and salts thereof. “Solvate” means a physical association of a compound of the invention with one or more solvent molecules. This physical association involves varying degrees of ionic and covalent bonding, including hydrogen bonding. In certain instances, the solvate will be capable of isolation. “Solvate” encompasses both solution-phase and isolatable solvates. Non-limiting examples of suitable solvates include ethanolates, methanolates and the like. “Hydrate” is a solvate wherein the solvent molecule is water.

The present invention also includes polymorphs of the compounds of the present invention and salts thereof. The term “polymorph” refers to a particular crystalline state of a substance, which can be characterized by particular physical properties such as X-ray diffraction, IR spectra, melting point, and the like.

### Additional Combinations

According to some embodiments, the present invention relates to a composition comprising a combination or conjugate comprising a plurality of compounds selected from (i) an anti-inflammatory compound or a fragment thereof; (ii) an anti-oxidant scavenger compound or a fragment thereof; (iii) a polyketide antibiotic compound or a fragment thereof; (iv) a metal chelating compound or a fragment thereof; and (v) a blood-brain-barrier targeting compound or a fragment thereof, for use in the treatment and/or prevention of a brain damage resulting from a brain injury. According to other embodiments, the combination is used in the treatment and/or prevention of a neurodegenerative disease. In accordance with these embodiments, the separate components, i.e., anti-inflammatory compound, an anti-oxidant compound, a polyketide antibiotic compound, a metal chelating compound, and a blood-brain-barrier targeting compound may not be covalently bound, or only a subset of said components may be covalently bound to form a conjugate, which is used in combination with the other components. The separate components may be administered in the same pharmaceutical composition, or in separate compositions intended for concomitant or sequential administration, in any order or dosing schedule apparent to a person of skill in the art. In addition, one compound may have multiple therapeutic functionalities such that the total number of components incorporated in the combination may be e.g., two, three, four, or five, at least two of which are covalently bound to form a conjugate.

According to some embodiments, the composition of the present invention may be administered to a subject in need as a prodrug. The prodrug often offers advantages such as solubility, tissue compatibility and/or delayed release. The term “pro-drug”, as used herein, refers to any component in the composition that when administered generates the biologically active “drug”/composition as a result of spontaneous chemical reaction(s), enzyme catalysed chemical reaction(s), and/or metabolic chemical reaction(s). The prodrug and derivatives thereof may be prepared by methods and techniques known to those of skill in the art.

As used herein, the term “administering” refers to bringing in contact with a composition of the present invention. Administration can be accomplished to cells or tissue cultures, or to living organisms, for example humans. In one embodiment, the present invention encompasses administering the compositions of the present invention to a human subject.

A “therapeutic” or “effective” treatment is a treatment administered to a subject who exhibits signs of pathology for the purpose of diminishing or eliminating those signs. A “therapeutically effective amount” or an “effective amount” of a composition of the invention is that amount of composition which is sufficient to provide a beneficial effect to the subject to which the compound is administered.

Non-limiting examples of anti-inflammatory compounds that are useful in the present invention include acemetacin, acetyl salicylic acid, alclofenac, alminoprofen, azapropazone, benorylate, benoxaprofen, bucloxic acid, carprofen, choline magnesium trisalicylate, clidanac, clopinac, dapsone, diclofenac, diflunisal, droxicam, etodolac, fenoprofen, fenbufen, fenclofenec, fentiazac, floctafenine, flufenisal, flurbiprofen, (r)-flurbiprofen, (s)-flurbiprofen, furofenac, feprazone, flufenamic acid, fluprofen, ibufenac, ibuprofen, indoprofen, isoxepac, isoxicam, ketoprofen, ketorolac, miroprofen, piroxicam, meloxicam, mefenamic, mefenamic acid, meclofenamic acid, meclofen, nabumetone, niflumic acid, oxaprozin, oxipinac, oxyphenbutazone, phenylbutazone, podophyllotoxin derivatives, proglumetacin, piprofen, pirprofen, prapoprofen, salicylic acid, salicylate, sudoxicam, suprofen, sulindac, tenoxicam, tiaprofenic acid, tiopinac, tioxaprofen, tolfenamic acid, tolmetin, zidometacin, zomepirac, and 2-fluoro-a-methyl[1 ,1′-biphenyl]-4-acetic acid, 4-(nitrooxy)butyl ester, butylated hydroxyanisole, butylated hydroxytoluene, propyl gallate, tert-butyl hydroquinone (TBHQ), Gossypol, Tocopherols and tocotrienols, among others. Each possibility represents a separate embodiment of the invention.

Non-limiting examples of anti-oxidant compounds include chalcone (such as butein), flavanol (such as catechins and epicatechins), flavone (such as apigenin, baicalein, chrysin, diosmin, luteolin, scutellarein, tangeritin, and wogonin), flavonol (such as quercetin, kaempferol, myricetin, fisetin, isorhamnetin, pachypodol, and rhamnazin), hydroxycinnamic acids (such as caffeic acid, cichoric acid, chlorogenic acid, caftaric acid, coumaric acid, coutaric acid, diferulic acids, fertaric acid, and ferulic acid) and stilbenoids (such as piceatannol) among others. Anti-oxidants may be of a synthetic or natural origin. Each possibility represents a separate embodiment of the invention.

Non-limiting examples of tetracycline antibiotic compounds include doxycycline, minocycline, chlortetracycline, oxytetracycline, demeclocycline, lymecycline, meclocycline, methacycline, rolitetracycline and tigecycline among others. Each possibility represents a separate embodiment of the invention.

Non-limiting examples of metal chelators include phosphoric acid, citric acid, ascorbic acid, ethylene diamine tetra acetate (EDTA).

Metal ions that can be chelated by the conjugates of the invention are, e.g., iron, zinc and copper, which are required to maintain normal function of the nervous system. These metal ions are transported into the brain, through the blood-brain barrier (BBB), where they are under tight metabolic control. In case of TBI, a breakdown of the BBB may occur, leading to increased BBB permeability. The influx and accumulation of these metals can initiate the production of reactive oxygen species depleting the brain's anti-oxidant defense systems, and activate microglial cells resulting in the release of inflammatory mediators that exacerbate the adverse effects of TBI.

In Alzheimer's disease, studies have shown that changes in the zinc and copper ions levels have been associated with the illness's neuropathy. It has been suggested that the toxic accumulation of beta amyloids, is due in part to excess binding of these metal ions which are abundant in the regions which are most affected. Thus, metal chelation is a component in the composition of the present invention for the treatment and/or prevention of a neurodegenerative disease or condition and brain injury.

The term “metal chelating compound” as used herein, refers to an organic compound that binds with and removes free metal ions from solution. The chelating agent may be of natural or synthetic source. Non-limiting examples of chelating agents include: synthetic chelating agents such as desferrioxamine, EDTA, and d-penicillamine, or natural chelating agents such as lactoferrin, inositol hexaphosphate (IP6), quercetin, catechin, ferulic acid, curcumin, ellagic acid, hydroxytyrosol, anthocyanidin among others. Each possibility represents a separate embodiment of the invention.

Non-limiting examples for solubility enhancer moieties include ionizable or polar neutral groups, such as ether, tertiary amine, phosphonium, phosphonite, nitro, sulfoxide, sulfonate, carbonate, carbamate, ester, amide, amino acid and sugar moiety among others. Each possibility represents a separate embodiment of the invention.

Non-limiting examples of a blood-brain-barrier targeting compound include glucose, pentose, allose, altrose, mannose, gulose, idose, galactose, talose, psicose, fructose, sorbose, tagatose, ribose, arabinose, xylose, lyxose, ribulose, xylulose, deoxyribose, fucose, rhamnose.

### Pharmaceutical Compositions

Although the composition of the present invention can be administered alone, it is contemplated that these compositions may be administered in a pharmaceutical composition containing the composition of the invention together with a pharmaceutically acceptable carrier and/or excipient.

According to some embodiments, the pharmaceutical compositions of the present invention can be formulated for administration by a variety of routes including oral, sublingual, buccal, rectal, vaginal, transdermal, parenteral (subcutaneous, intraperitoneal, intravenous, intra-arterial, transdermal and intramuscular), topical, intranasal, dialysis or inhalation among others. Each possibility is a separate embodiment of the invention.

Such pharmaceutical compositions are prepared in a manner well known in the pharmaceutical art and comprise as an active ingredient a composition of the present invention as described hereinabove, and a pharmaceutically acceptable excipient and/or carrier. The term “pharmaceutically acceptable” means approved by a regulatory agency of the Federal or a state government or listed in the U.S. Pharmacopeia or other generally recognized pharmacopeia for use in animals and, more particularly, in humans.

During the preparation of the pharmaceutical compositions according to the present invention the active ingredient is usually mixed with a carrier and/or excipient, which may be a solid, semi-solid, or liquid material. The pharmaceutical compositions are preferably suited for oral administration, in which case they can be in the form of tablets, pills, capsules, pellets, polymers, granules, powders, lozenges, sachets, cachets, elixirs, suspensions, dispersions, emulsions, solutions, syrups, aerosols (as a solid or in a liquid medium), soft and hard gelatin capsules, suppositories, sterile injectable solutions and sterile packaged powders.

The carriers may be any of those conventionally used. The choice of carrier will be determined by the particular method used to administer the pharmaceutical composition. Some examples of suitable carriers include lactose, glucose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water and methylcellulose. Other pharmaceutical carriers can be sterile liquids, such as water, alcohols (e.g., ethanol) and lipid carriers such as oils (including those of petroleum, animal, vegetable or synthetic origin, such as peanut oil, soybean oil, mineral oil, sesame oil and the like), phospholipids (e.g. lecithin), polyethylene glycols, glycerine, propylene glycol or other synthetic solvents. Water is a preferred carrier when the pharmaceutical composition is administered intravenously. Saline solutions and aqueous dextrose and glycerol solutions can also be employed as liquid carriers, particularly for injectable solutions.

The formulations can additionally include lubricating agents such as talc, magnesium stearate, and mineral oil; wetting agents, surfactants, emulsifying and suspending agents; preserving agents such as methyl- and propylhydroxybenzoates; sweetening agents; flavoring agents, colorants, buffering agents (e.g., acetates, citrates or phosphates), disintegrating agents, moistening agents and agents for the adjustment of tonicity such as sodium chloride. Fatty acids can also be included.

Alternative formulations include immediate-release formulations, slow-release formulations, controlled-release formulations, delayed-release formulations and the like, as are known in the art.

The amount of a composition of the invention that will be effective in the treatment of a particular disorder or condition, including TBI, ABI and neurodegenerative diseases, will depend on the nature of the disorder or condition, and can be determined by standard non-clinical or clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. A preferred dosage will be within the range of 0.01-1000 mg/kg of body weight, more preferably, about 0.1 mg/kg to about 100 mg/kg and even more preferably about 1 mg/kg to about 10 mg/kg. Effective doses may be extrapolated from dose-response curves derived from in vitro or animal model test bioassays or systems.

### Therapeutic Use

As contemplated herein, the conjugates compositions and pharmaceutical compositions of the invention are particularly useful for preventing and treating brain damage resulting from a brain injury.

Thus, according to one aspect, the present invention provides a method for treating or preventing brain damage resulting from a brain injury, by administering to a subject in need thereof an effective amount of a conjugate or composition as described herein. In some embodiments, the composition is administered in a pharmaceutical composition.

According to yet another aspect, the present invention relates to the use of a conjugate or composition as described herein, or a pharmaceutical composition comprising such conjugate or composition, in the manufacture of a medicament for treating or preventing brain damage resulting from brain injury.

According to some embodiments, the brain injury is traumatic brain injury (TBI). In accordance with this embodiment, the brain damage is a secondary brain damage resulting from TBI. TBI is characterized by a series of primary and secondary damage events that ultimately lead to deleterious effects at the cellular level as well as long term effects at the systemic level. Primary damage includes damage to the BBB and cellular membranes, with resulting axon transport destruction and blood vessel leakage. Secondary damage includes the failure of ATP production, ion imbalance (Na⁺, K⁺), glutamate and excitatory amino acid release, influx of Ca⁺⁺ and other ions, free radial production/ROS, DNA damage, oxidative stress & nitric oxide generation, activation of caspases, lysosomal rupture, activation of hydrolytic pathways, activation of inflammatory pathways, metal toxicity, lipid peroxidation, phosphatase activation and proteolysis. Effects at the cellular level include neuro-inflammation, necrosis, cell death and non-neuronal cell loss. These all can lead to long term effects such as depression, seizures, suicidal tendencies, delusion (paranoia), and increased risk of neurodegenerative conditions such as Alzheimer's and Parkinson's Disease. The multi-function compounds of the present invention target multiple mechanisms of action that simultaneously intervene in multiple different biochemical and physiological pathways. As such, the composition of the invention is more effective than single agent therapy at treating or preventing the primary and secondary damage associated with brain injury or its related long-term effects.

According to some embodiments, the brain injury is an acquired brain injury (ABI). In accordance with this embodiment, the acquired brain injury results from stroke, surgery, brain tumour, injury caused by brain tumour removal, surgery, infection, chemical and/or toxic poisoning, hypoxia, ischemia, encephalopathy, substance abuse, deceleration/blast injury or a combination thereof.

As contemplated herein, the conjugates and compositions of the invention are also useful for preventing and treating secondary brain damage resulting from TBI. Thus, the conjugates or compositions of the invention are useful, e.g., for treating soldiers in the battlefield, especially soldiers who have suffered TBI. For example, secondary brain damage due to TBI can be treated or prevented by administering the conjugates compositions of the invention to soldiers, or by supplying paramedics in the battlefield or at the site of terrorist attack with the compositions of the invention, so that the compositions can be administered on site as soon as possible after the soldier has suffered TBI. The compositions of the invention are also useful for civilians who are victims of violent crimes, including but not limited to, terrorist attacks. This may reduce the incidence of disability presently occurring in the aftermath of TBI. The compositions of the invention are also useful for individuals suffering from brain injury due to domestic occurrences, such as automobile accidents, sports injuries, household accidents, child abuse, gun-shot wounds, concussion/s, explosions, blasts, etc., including their consequences such events such as disability and epilepsy.

According to yet another aspect, the present invention provides a method for treating or preventing a neurodegenerative disease or condition, by administering to a subject in need thereof an effective amount of a conjugate or composition as described herein. In some embodiments, the conjugate or the composition is administered in a pharmaceutical composition.

According to yet another aspect, the present invention relates to the use of a conjugate or a composition as described herein, or a pharmaceutical composition comprising such conjugate or composition, in the manufacture of a medicament for the treatment or prevention of a neurodegenerative disease or condition.

According to some preferred embodiments, the neurodegenerative disease or condition is selected from the group consisting of Alzheimer's Disease, Multiple System Atrophy (MSA), Parkinsonism (i.e., Parkinson's syndrome, atypical Parkinson's, or secondary Parkinson's, including Parkinson's Disease), and Amyotrophic Lateral Sclerosis (ALS). Each possibility represents a separate embodiment of the invention.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Example 1: Synthesis of conjugate (1)

1. Initially, on a Wang resin solid support a scaffold is synthesized, wherein A, B and C are different orthogonal protecting groups which are removed stepwise.

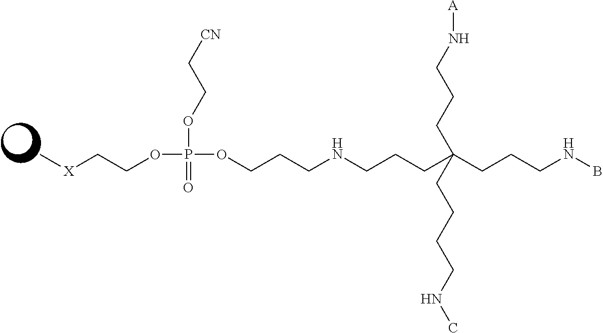

2. Removal of protecting group A:

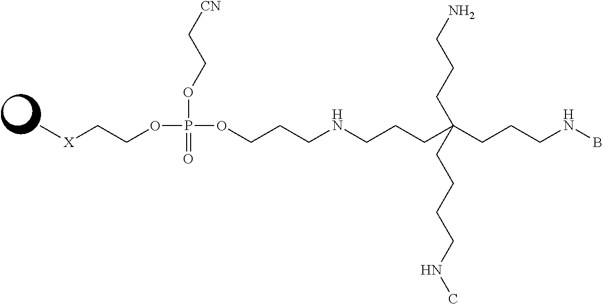

3. Attachment of the antioxidant:

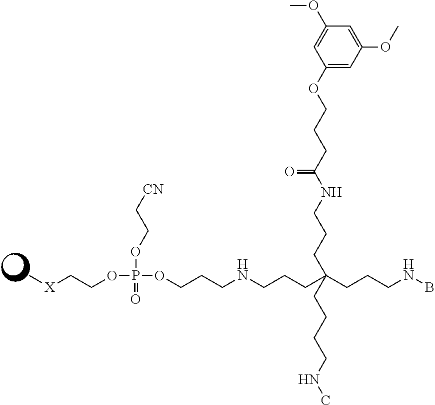

4. Removal of protecting group B:

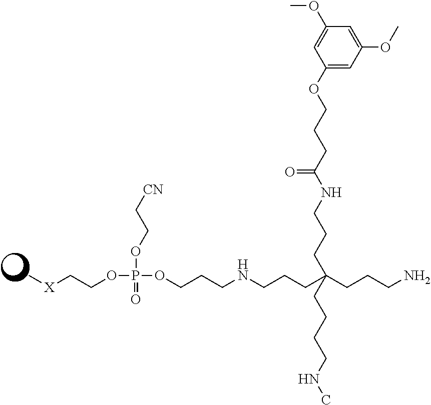

5. Attachment of the anti-inflammatory compound:

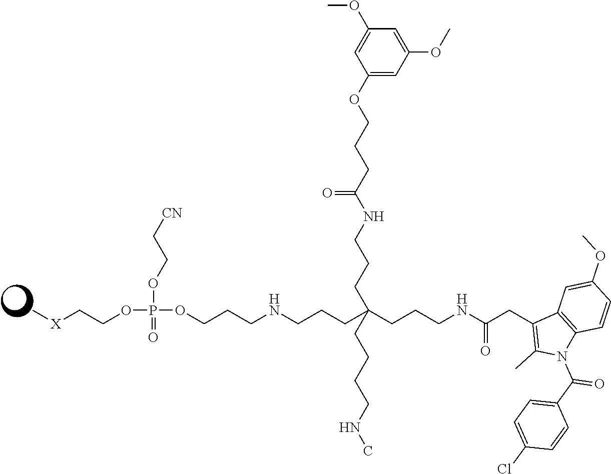

6. Removal of protecting group C:

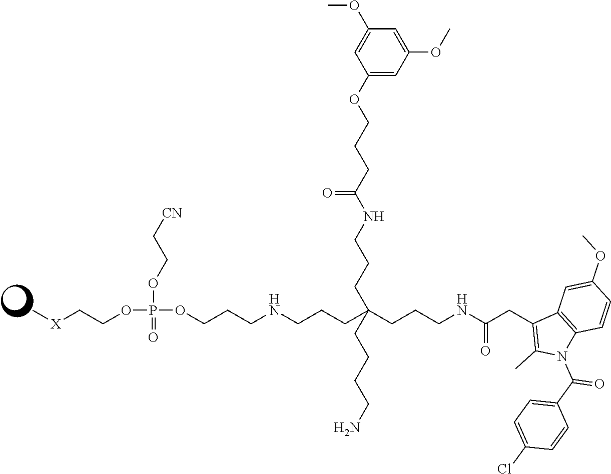

7. Attachment of the metal chelator:

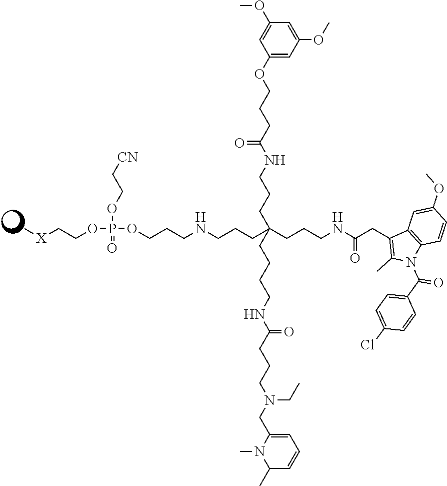

8. Detachment of the molecule from the solid support:

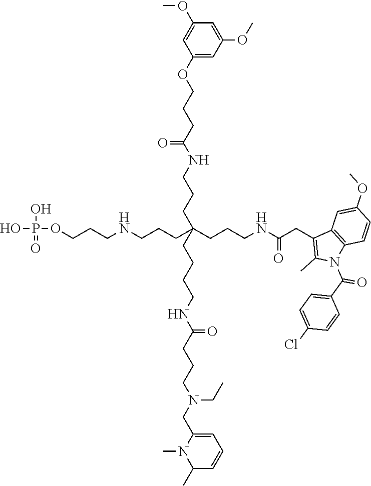 [SMILES: CCN(CCCC(=O)NCCCCC(CCCNCCCOP(=O)(O)O)(CCCNC(=O)CCCOc1cc(OC)cc(OC)c1)CCCNC(=O)Cc1c(C)n(C(=O)c2ccc(Cl)cc2)c2ccc(OC)cc12)CC1=CC=CC(C)N1C]

9. Attachment of the antibiotic:

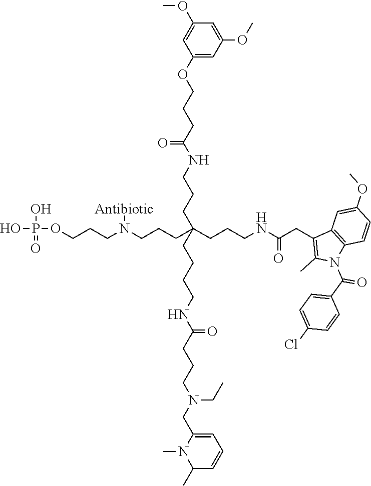

10. The last step is demethylation of the methyl ether groups:

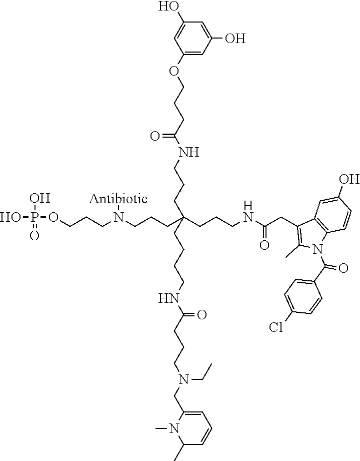

The molecular weight of this conjugate is in the range of 1200-1700 Da. The conjugate is purified by HPLC to 95% purity.

### Example 2: Synthesis of Compound (3) (2,3-dihydroxy-N-(2-(2-(3-hydroxy-2-(((1R,2R,3S,4S)-2,3,4,5-tetrahydroxycyclohexyl)methoxy) phenoxy) ethoxy)phenyl) benzamide), Compound (4) (2,3-dihydroxy-N-(2-(2-(2-(((2R,3S,4S,5R)-3,4,5,6-tetrahydroxytetrahydro-2H-pyran-2-yl)methoxy)phenoxy)ethoxy)phenyl)benzamide) and Compound (5) N-(2-(2-(3,4-dihydroxy-2-(((2R,3S,4S,5R)-3,4,5,6-tetrahydroxytetrahydro-2H-pyran-2-yl)methoxy)phenoxy)ethoxy)phenyI)-2,3-dihydroxybenzamide.

Compound (3) (2,3-dihydroxy-N-(2-(2-(3-hydroxy-2-(((1R,2R,3S,4S)-2,3,4,5-tetrahydroxycyclohexyl)methoxy) phenoxy) ethoxy)phenyl) benzamide) and compound (4) (2,3-dihydroxy-N-(2-(2-(2-(((2R,3S,4S,5R)-3,4,5,6-tetrahydroxytetrahydro-2H-pyran-2-yl)methoxy)phenoxy)ethoxy)phenyl)benzamide) may be prepared in accordance with the following representative process.

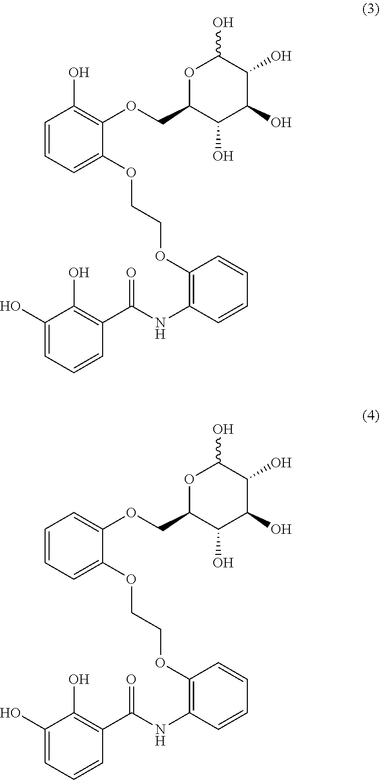 [SMILES: O=C(Nc1ccccc1OCCOc1cccc(O)c1OC[C@H]1OC(O)[C@H](O)[C@@H](O)[C@@H]1O)c1cccc(O)c1O ; O=C(Nc1ccccc1OCCOc1ccccc1OC[C@H]1OC(O)[C@H](O)[C@@H](O)[C@@H]1O)c1cccc(O)c1O]

In stage 1 (Scheme 1), 2-nitrophenol is reacted with ethylene dibromide to produce 1-(2-bromoethoxy)-2-nitrobenzene, which is subsequently reacted with benzene-1,2,3-triol (R¹═OH) or 1,2-dihydroxybenzene (R¹═H).

### Stage 1

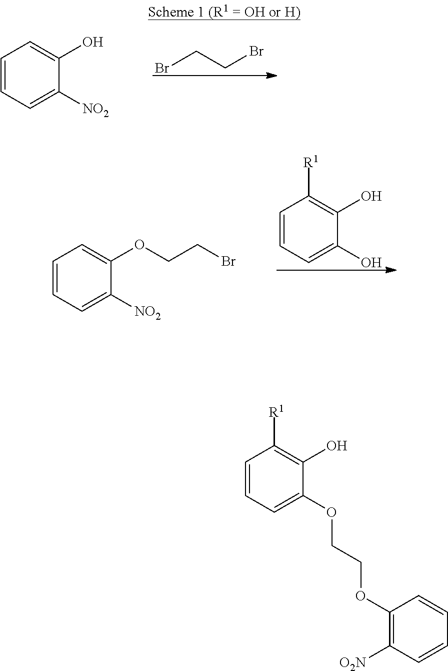

In stage 2 (Scheme 2), the sugar moiety (saccharide) is prepared. Using glucose as a representative sugar, Scheme 2 shows reaction of glucose methyl ether with PPh₃ and Br₂ to replace the C-6 OH with a Br. The remaining free hydroxyls at C-3, C-4 and C-5 may protected with Benzyl (R²═Bn) groups, which are removable by hydrogenation as known in the art. Additional protecting groups suitable for this reaction are benzoyl groups (R²═Bz), which are removable by base-catalyzed hydrolysis, or acetyl groups (R²═Ac), which are removable by acid-catalyzed hydrolysis. In another embodiment, the free hydroxyls at C-3, C-4 and C-5 are unprotected during the reaction.

### Stage 2

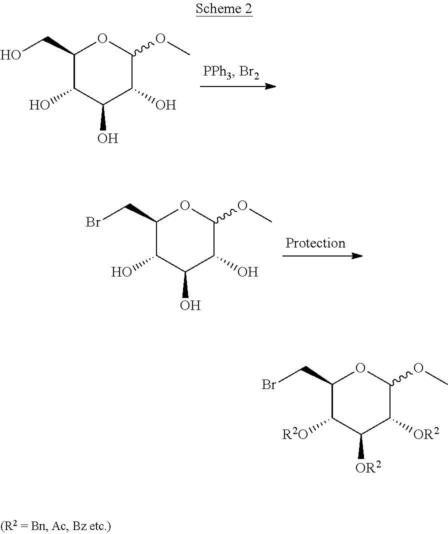

In stage 3 (Scheme 3), the products of stage 1 and 2 are coupled. The protecting groups (R²) are removed. When R²═Bn, this step conveniently also includes reduction of the nitro group to an amine, generating an aniline derivative which is coupled to dihydroxybenzoic acid. If R² is other than Bn, a further reduction step of the nitro group to the corresponding amino group is performed. Acid deprotection of the methyl ether group with HCI produces the compounds of formula (3) or (4) in good yield.

### Stage 3

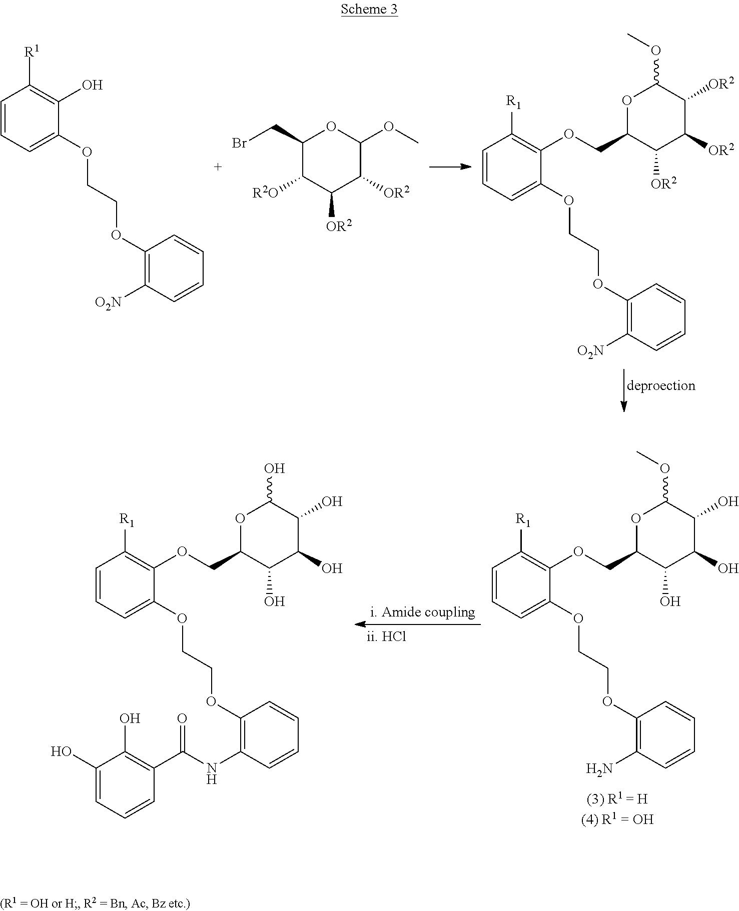

Compound (5) (N-(2-(2-(3,4-dihydroxy-2-(((2R,3S,4S,5R)-3,4,5,6-tetrahydroxytetrahydro-2H-pyran-2-yl)methoxy)phenoxy)ethoxy)phenyl)-2,3-dihydroxybenzamide) can be prepared in a similar manner starting from benzene-1,2,3,4-tetraol or a protected derivative thereof as a starting material in Stage 1.

### Example 3: Alternative Route 1 to synthesize compounds (3), (4) and (5):

An alternative route for the synthesis of the compounds 3 and 4 of the invention is provided in Scheme 4. In accordance with this route, 3-methoxybenzene-1,2-diol (R³═OMe) or 1,2-dihydroxybenzene (R³═H) is coupled with a protected 6-bromo glucose derivative and then 1-(2-bromoethoxy)-2-nitrobenzene to yield a protected intermediate which is initially then hydrogenated/reduced and deprotected, then coupled with DHB and finally undergoes acid deprotection of the methyl ether group with HCI.

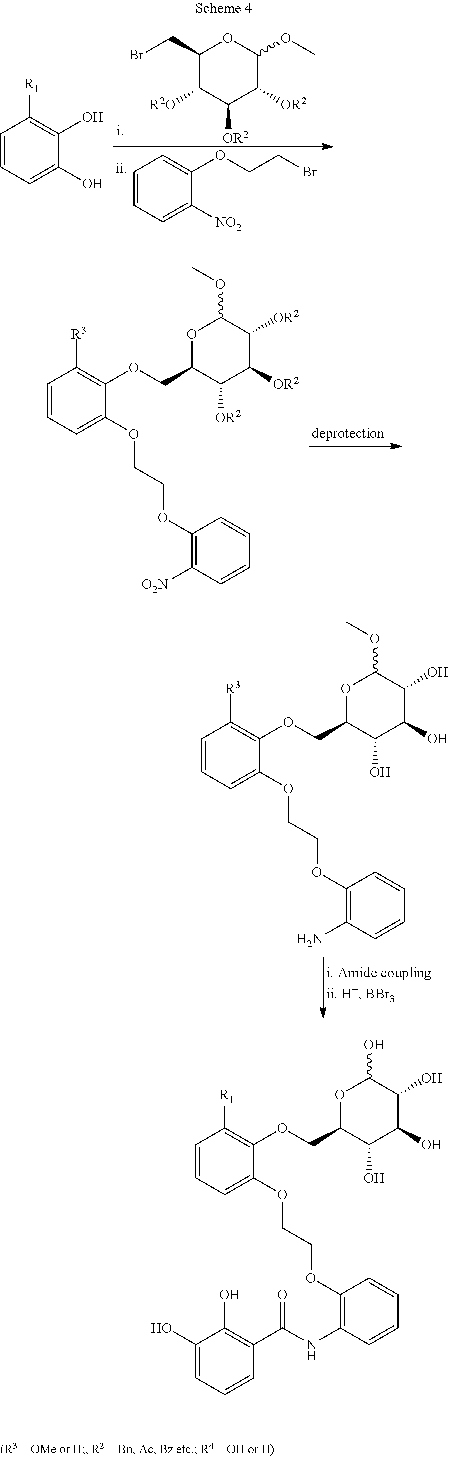

Compound (5) (N-(2-(2-(3,4-dihydroxy-2-(((2R,3S,4S,5R)-3,4,5,6-tetrahydroxytetrahydro-2H-pyran-2-yl)methoxy)phenoxy)ethoxy)phenyl)-2,3-dihydroxybenzamide) can be prepared in a similar manner starting from benzene-1,2,3,4-tetraol or a protected derivative thereof as a starting material.

### Example 4: Alternative Route 2 to synthesize compounds (3), (4) and (5):

Compound (3) may also be prepared in accordance with the following process. There are two alternative hydroxy protecting groups used: 1) methoxymethyl (MOM) (steps 1, 2A, 3, 4A, 5A, 6A and 7A); and 2) methyl (steps 1, 2B, 2B, 3, 4B, 5B, 6B and 7B). It is apparent to a person of skill in the art that any other hydroxy protecting groups as described herein may be used without deviating from the scope and spirit of the invention.

Step 1: 2-nitrophenol is reacted with ethylene dibromide to produce 1-(2-bromoethoxy)-2-nitrobenzene.

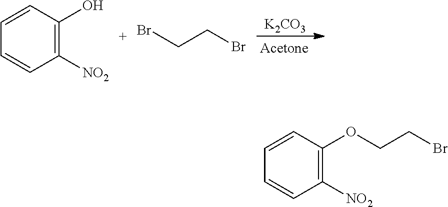

Step 2A: 1-(2-bromoethoxy)-2-nitrobenzene obtained in step 1 is reacted with 3-(methoxymethoxy)benzene-1,2-diol (one of the hydroxyls in benzene-1,2,3-triol is protected with a methoxymethyl (MOM) group) to yield 2-(methoxymethoxy)-6-(2-(2-nitrophenoxy)ethoxy)phenol

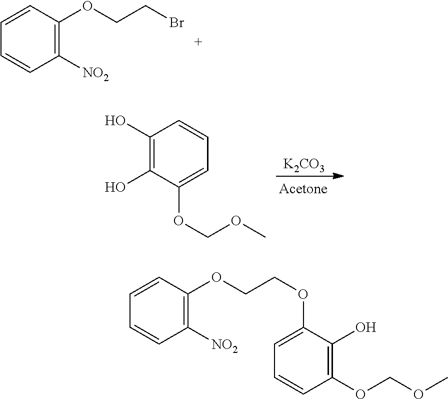

Step 2B: Alternatively, a methyl ether can be used as a protecting group, instead of the MOM group:

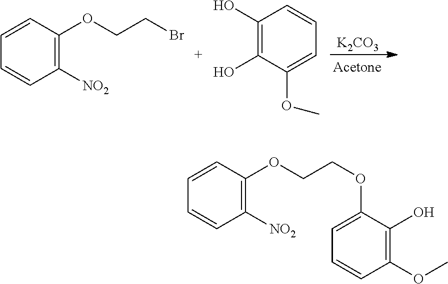

Step 3: the sugar moiety is prepared. Using glucose as a representative sugar, this step involves reaction of glucose methyl ether with PPh₃ and Br₂ to replace the C-6 OH with a Br. The remaining free hydroxyls at C-3, C-4 and C-5 are protected with 3,4-dihydropyranyl groups, which are removable by acid deprotection as known in the art. In another embodiment, the free hydroxyls at C-3, C-4 and C-5 are unprotected during the reaction.

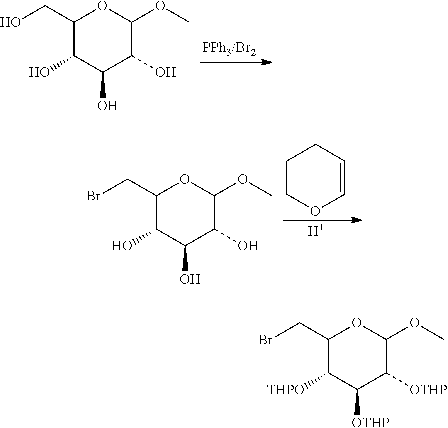

Step 4A: the products of step 2A and 3 are coupled.

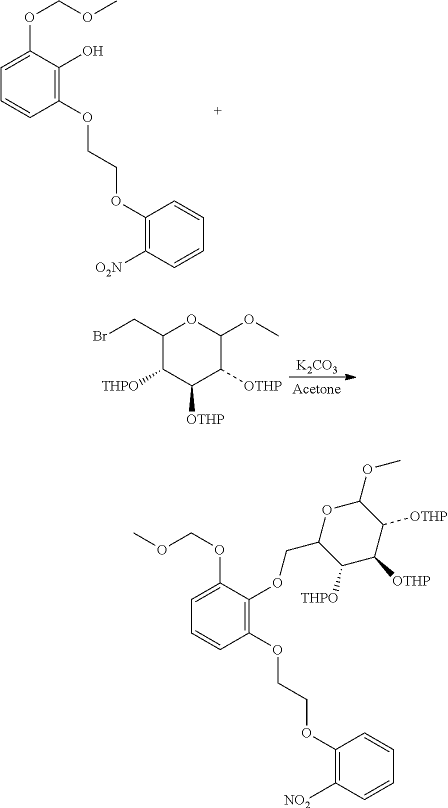

Step 4B: Alternatively, the products of step 2B and 3 are coupled.

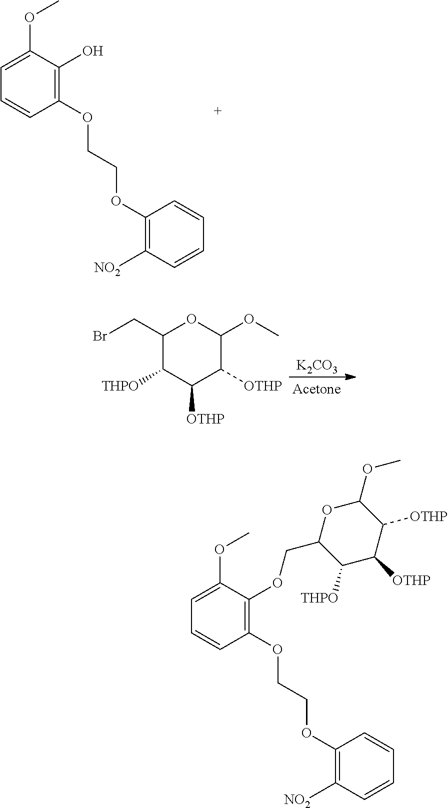

Step 5: this step includes reduction of the nitro group to an amine by a hydrogenation reaction, generating an aniline derivative. The reaction can be conducted with the product of step 4A (MOM protecting group) or 4B (methyl protecting group).

Step 5A (MOM protecting group):

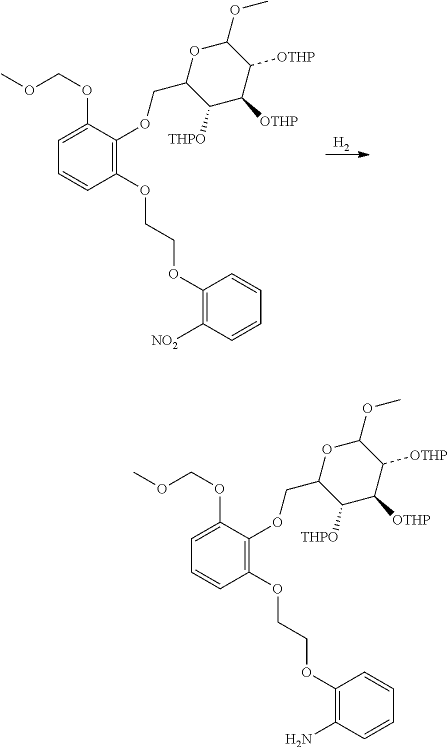

Step 5B: methyl protecting group:

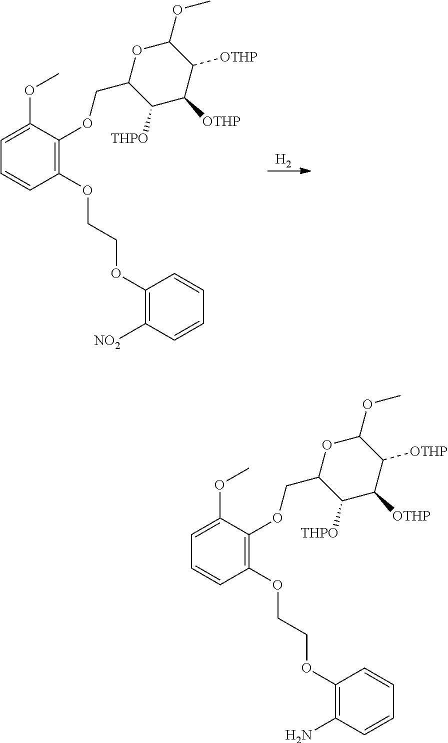

Step 6: the aniline derivative obtained in step 5 is coupled to 2,3 dihydroxybenzoic acid using the coupling reagent DCC. The reaction can be conducted with the product of step 5A (MOM protecting group) or 5B (methyl protecting group).

Step 6A (MOM protecting group):

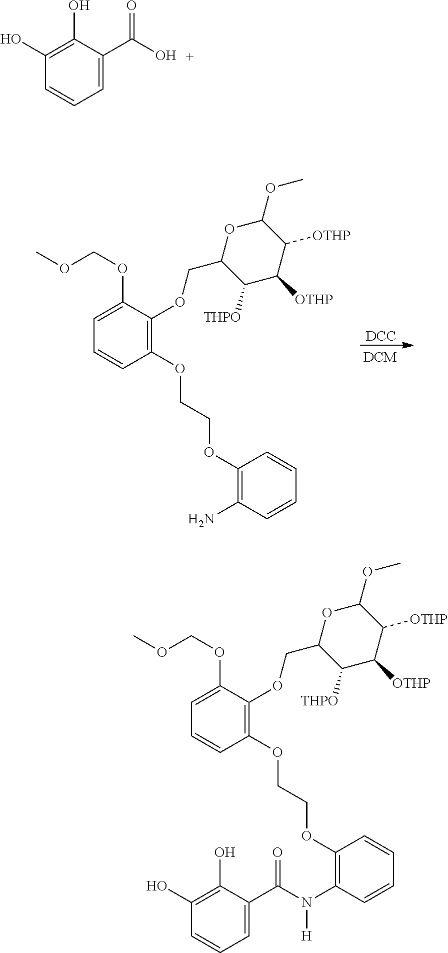

Step 6B (methyl protecting group):

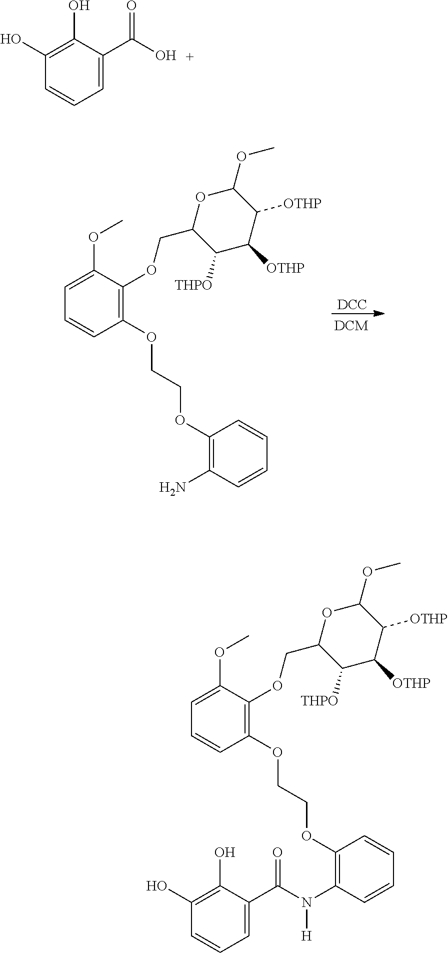

Step 7: acid deprotection removes the various hydroxyl protecting groups rendering the compound of formula (3) in good yield. The reaction can be conducted with the product of step 6A (MOM protecting group) or 6B (methyl protecting group).

Step 7A (MOM protecting group):

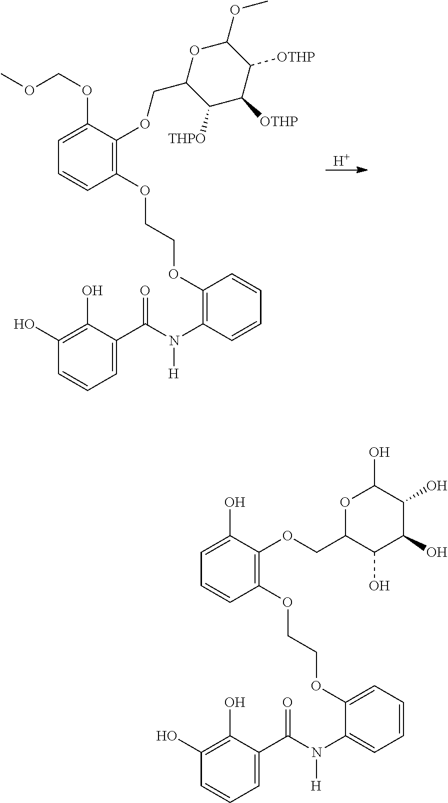

Step 7B (methyl protecting group):

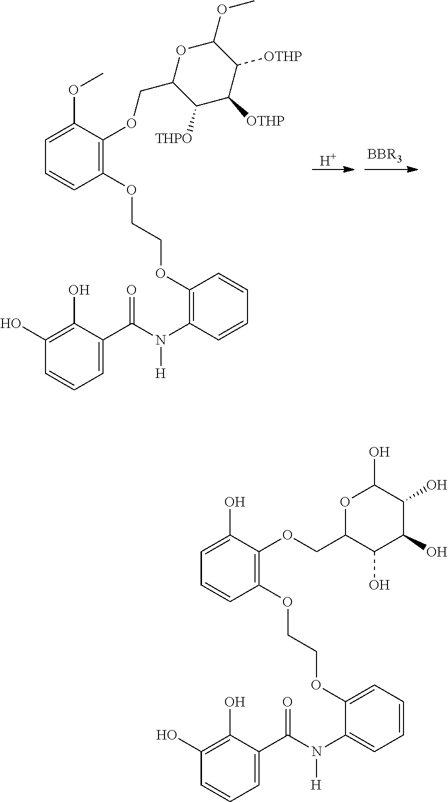

Compound (4) can be prepared in a similar manner starting from 1,2-dihydroxy benzene as a starting material. Compound (5) can be prepared in a similar manner starting from protected benzene-1,2,3,4-tetraol as a starting material.

### Example 5: The Effect of Compositions on TBI In Vivo

The effect of the compositions of the present invention in treating TBI is determined in a mouse model subjected to a close head injury. Briefly, closed head injury is carried by a weight-drop onto the exposed skull. A midline longitudinal incision is performed, when the skull is exposed, the left anterior frontal area is identified. A Teflon-tipped cone (2-mm diameter) is placed 1 mm lateral to the midline, in the mid-coronal plane. While the head is held in place manually, a 95 g weight is dropped on the cone from a height of 10 cm for severe and 6 cm for mild trauma, resulting in a focal injury to the left hemisphere.

Eight groups of C57BL/6 mice are subcutaneously administered with vehicle (control; n=8), anti-inflammatory compound (treatment; n=8), anti-oxidant compound (treatment; n=8), metal chelating compound (treatment; n=8), polyketide antibiotic compound (treatment; n=8), DHB alone (treatment; n=8), compound (1) of the invention (treatment; n=8) or one or more combinations of the above components or a conjugate comprising same (treatment; n=8). These compounds are given from day eight, three times a week.

The severity of injury is based on the assessment of the Neurological Severity Score (NSS) at 1 h post challenge for baseline (reflects the severity of the initial injury) and seven days after trauma. The following parameters are examined: morbidity and mortality (daily); body weight (twice a week); clinical observations (twice a week); open field (OF), elevated maze and social recognition tests are carried at baseline (Day-3), on Day 7 (before treatment) and on Day 30 (total of three times).

Five weeks post challenge, the mice are sacrificed. Terminal bleeding and plasma preparation are kept at −20° C. for further cytokines and S-100-B levels test. The cytokines' profile is assessed by Multiplex ELISA. S100-B, a marker that reflects the presence of neuropathological conditions including TBI, is quantified by ELISA. Finally, the brains are removed and processed for histopathological evaluation using H&E staining.

### Example 6: Synthesis of N-{o-[2-(2-{[(2R,3S,4S,5R,6R)-3,4,5,6-Tetrahydroxytetrahydro-2Hpyran-2-yl]methoxy}-3,4-dihydroxyphenoxy)ethoxy]phenyl}2,3-dihydroxybenzamide (19)

The compound of formula 19 was successfully synthesized via the 18 -step synthesis starting from 2,3-dihydrobenzaldehyde 1 using the route shown below.

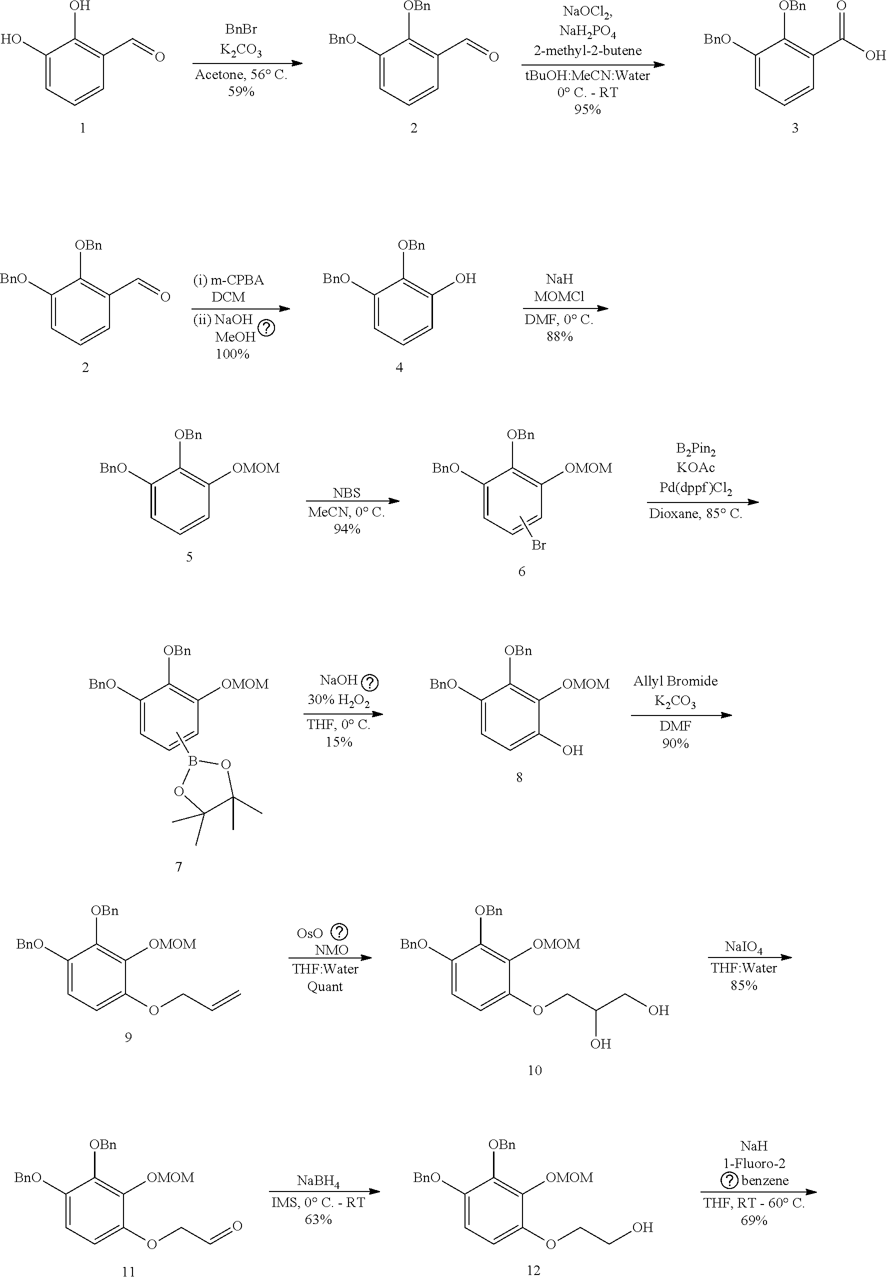

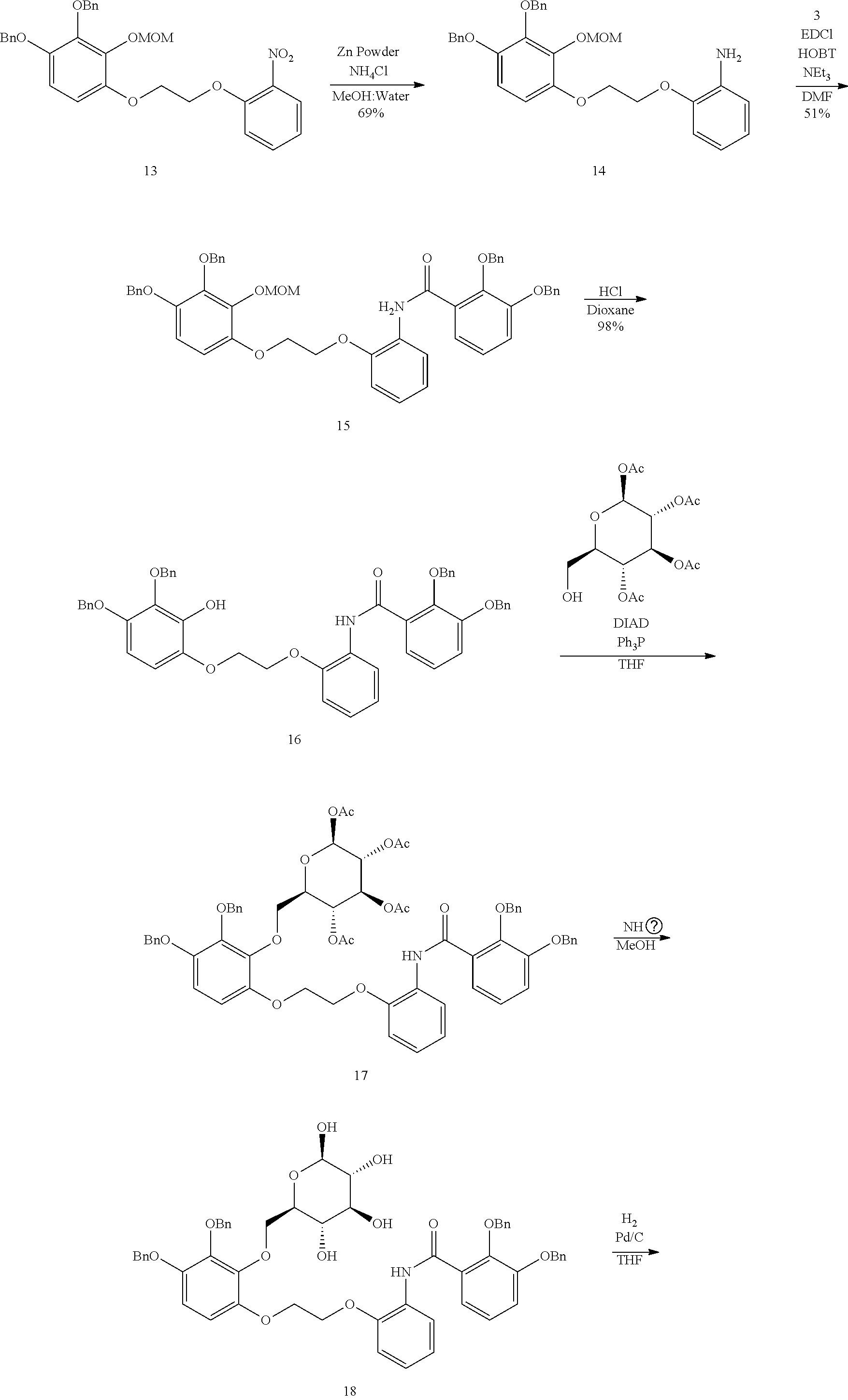

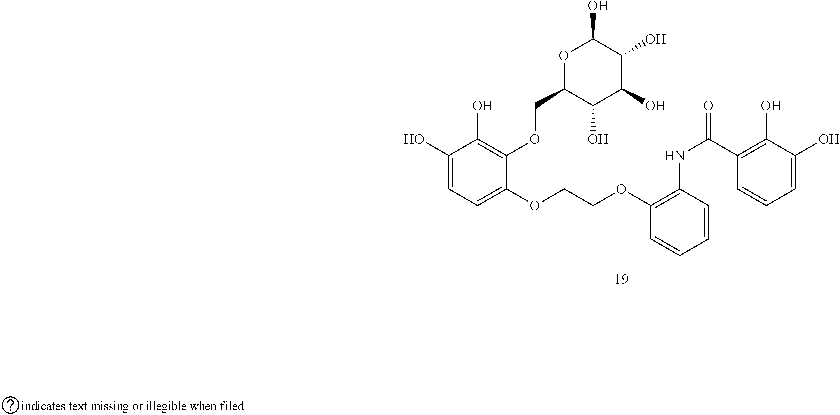 [SMILES: O=C(Nc1ccccc1OCCOc1ccc(O)c(O)c1OC[C@H]1O[C@@H](O)[C@H](O)[C@@H](O)[C@@H]1O)c1cccc(O)c1O]

### Synthesis of 2,3-Bis(benzyloxy)benzaldehyde (2)

Benzyl bromide (56.9 mL, 0.478 mol) was added dropwise to a suspension of 2,3-dihydrobenzaldehyde (30.0 g, 0.217 mol) and potassium carbonate (66.1 g, 0.478 mol). Reaction mixture was then heated at 56 ° C. for 16 hours. Then cooled to room temperature, filtered and filtrate concentrated in vacuo. EtOAc (550 mL) and water (300 mL) were added to crude residue and separated, organic phase dried over sodium sulfate, filtered and solvent removed in vacuo to give gummy solid. Solid triturated with ether to obtain 2,3-Bis(benzyloxy)benzaldehyde (28.9 g) as a white solid. Trituration liquors were concentrated and purified by dry flash chromatography (0-5% EtOAc in petroleum ether), fractions combined and solvent removed in vacuo to give obtain 2,3-Bis(benzyloxy)benzaldehyde (3.4 g). Mixed fractions were combined and solvent removed in vacuo, then triturated with IPA to obtain another batch of 2,3-Bis(benzyloxy)benzaldehyde (8.45 g) as a white solid. Batches combined led to 2,3-Bis(benzyloxy)benzaldehyde (40.8 g, 59%) as a white solid. LCMS 341.2 [M+Na]⁺ 97% Purity. ¹H NMR (500 MHz, CDCl₃) δ 10.29 (d, J=0.9 Hz, 1H), 7.53−7.47 (m, 2H), 7.47−7.40 (m, 3H), 7.43−7.36 (m, 1H), 7.35 (s, 5H), 7.27 (dd, J=8.1, 1.6 Hz, 1H), 7.14 (td, J=8.0, 0.9 Hz, 1H), 5.23 (s, 2H), 5.22 (s, 2H).

Synthesis of 2,3-Bis(benzyloxy)benzoic acid (3)

2,3-Bis(benzyloxy)benzaldehyde (1.86 g, 5.84 mmol) was dissolved in t-BuOH (35 mL), MeCN (35 mL), 2-methyl-2-but-2-ene (18.5 mL, 175.2 mmol) was added. Reaction mixture was cooled to 0° C., NaOCl₂ (5.3 g, 58.4 mmol) and NaH₂PO₄ (5.1 g, 42.65 mmol) in water (10 mL) was then added dropwise. Reaction mixture was allowed to warm to room temperature and stirred for 2 hours. Reaction mixture was acidified to pH=1 and then extracted with EtOAc (3×50 mL). Combined organics were washed with water (75 mL), Brine (75 mL), dried over sodium sulfate, filtered and solvent removed in vacuo. Trituration of crude material with petroleum ether obtained 2,3-Bis(benzyloxy)benzoic acid (1.85 g, 95%) as a white solid. ¹H NMR (500 MHz, DMSO) δ 12.74 (s, 1H), 7.53−7.47 (m, 2H), 7.43−7.38 (m, 4H), 7.38−7.34 (m,2H), 7.34−7.30 (m, 3H), 7.23 (dd, J=7.8, 1.6 Hz, 1H), 7.16 (t, J=7.9 Hz, 1H), 5.20 (s, 2H), 5.01 (s, 2H).

### Synthesis of 2,3-Bis(benzyloxy)phenol (4)

A solution of 2,3-Bis(benzyloxy)benzaldehyde (20.3 g, 63.8 mmol) in DCM (250 mL) was cooled to 0° C., m-CPBA (21.0 g, 121.2 mmol) was then added portion wise, reaction mixture was then stirred at room temperature for 16 hours. Reaction mixture was filtered, filtrate then concentrated in vacuo. The concentrated residue was then dissolved in MeOH (250 mL), NaOH (5.56 g, 140.36 mmol) in water (7 mL) was added and stirred at room temperature for 1 hour. A second batch, limiting starting material (3.0 g, 9.42 mmol) was combined with the concentrated material. Reaction mixture was concentrated in vacuo. Sat. NH₄Cl (500 mL) and EtOAc (500 mL) were added, solid precipitated out and filtered. Phases were separated and aqueous re-extracted with

EtOAc (2×250 mL), combined organics washed with brine (200 mL), dried over sodium sulfate, filtered and solvent removed in vacuo. 2,3-Bis(benzyloxy)phenol (22.4 g, 99.8%) was obtained as a brown solid. LCMS 307.2 [M+H]⁺ 96% Purity. ¹H NMR (500 MHz, CDCl₃) δ 7.47 (d, J=7.1 Hz, 2H), 7.44−7.31 (m, 8H), 6.91 (t, J=8.2 Hz, 1H), 6.58 (ddd, J=8.5, 5.8, 1.4 Hz, 2H), 5.15 (s, 2H), 5.10 (s, 2H).

Synthesis of 1,2-Bis(benzyloxy)-3-methoxymethoxybenzene (5)

A suspension of 60% sodium hydride in mineral oil (2.43 g, 60.7 mmol) in DMF (35 mL) was cooled to 0° C. and 2,3-Bis(benzyloxy)phenol (17.7 g, 57.8 mmol) in DMF (35 mL) was added dropwise. The reaction mixture was then stirred at 0° C. for 40 minutes. MOMCI (4.81 mL, 63.6 mmol) was added dropwise and reaction stirred for a further 45 minutes. Water was added to quench reaction. Aqueous then extracted with EtOAc (3×200 mL), combined organics were then washed with water (200 mL) and Brine (200 mL). Then dried over sodium sulfate, filtered and solvent removed in vacuo. Residue was then dissolved in ether (200 mL) and washed with water (2×200 mL), Brine (100 mL), dried over sodium sulfate, filtered and solvent removed in vacuo. Crude material was purified by flash column chromatography (0-7% EtOAc in petroleum ether) fractions combined and solvent removed in vacuo. 1,2- Bis(benzyloxy)-3-methoxymethoxybenzene (17.37 g, 88%) was obtained as a colourless oil. LCMS 3541.2 [M+H]⁺ 98% Purity. ¹H NMR (500 MHz, CDCl₃) δ 7.47−7.28 (m, 10H), 6.95 (t, J=8.3 Hz, 1H), 6.80 (dd, J=8.4, 1.3 Hz, 1H), 6.69 (dd, J=8.3, 1.4 Hz, 1H), 5.16 (s, 2H), 5.11 (s, 2H), 5.07 (s, 2H), 3.49 (s, 3H).

Synthesis of a mixture of 1,2-Bis(benzyloxy)-4-bromo-3-methoxymethoxybenzene and 2,3- Bis(benzyloxy)-1-bromo-4-methoxymethoxybenzene (6)

NBS (8.33 g, 46.8 mmol) was added portion-wise to a solution of 1,2-Bis(benzyloxy)-3-methoxymethoxybenzene (16.4 g, 46.8 mmol) in acetonitrile (160 mL) cooled to 0° C., under atmosphere of N₂. After 4 hours, reaction mixture was concentrated to dryness and purified by dry flash chromatography (10% EtOAc in petroleum ether) fractions combined and solvent removed in vacuo to give mixture of 1,2-Bis(benzyloxy)-4-bromo-3-methoxymethoxybenzene and 2,3-Bis(benzyloxy)-1-bromo-4-methoxymethoxybenzene (18.85 g, 94%) as a brown oil. LCMS No ionisation.

Synthesis of mixture of 2-[3,4-Bis(benzyloxy)-2-methoxymethoxyphenyl]-4,4,5,5-tetramethyl-1,3,2-dioxaborolane and 2-[2,3-Bis(benzyloxy)-4-methoxymethoxyphenyl]-4,4,5,5-tetramethyl-1,3,2-dioxaborolane (7)

A suspension of mixture of brominated isomers (6) (12.9 g, 30 mmol), Bis(pinacolato)diboron (22.9 g, 90 mmol) and KOAc (14.7, 150 mmol) in dioxane (130 mL) was degassed with nitrogen for 10 minutes, Pd(dppf)Cl₂ (1.1 g, 1.5 mmol) was added and reaction mixture was heated to 85 ° C. for 16 hours under atmosphere of nitrogen. Reaction mixture was cooled to room temperature and filtered through celite. Water (100 mL) was added to the filtrate and then extracted with EtOAc (2×200 mL), combined organics were washed with brine (300 mL), dried over sodium sulfate, filtered and solvent removed in vacuo. Crude material was purified by dry flash chromatography (100% EtOAc in petroleum ether) fractions combinedand solvent removed in vacuo. 2-[3,4-Bis(benzyloxy)-2-methoxymethoxyphenyl]-4,4,5,5-tetramethyl-1,3,2-dioxaborolane and 2-[2,3-Bis(benzyloxy)-4-methoxymethoxyphenyl]-4,4,5,5-tetramethyl-1,3,2-dioxaborolane (30.4 g) was obtained as a brown oil. Impurities taken through to next step. LCMS 477.3 [M+H]⁺ 69% Purity.

Synthesis of 3,4-Bis(benzyloxy)-2-methoxymethoxyphenol (8)

A solution of the mixture of borylated isomers (7) (25 g, 52.5 mmol) in THF (400 mL) was cooled to 0° C. 1M NaOH (53 mL) was added dropwise, followed by dropwise addition of 30% hydrogen peroxide (53 mL). Reaction was stirred for 30 minutes at room temperature. A second batch, limiting starting material (5.0 g, 10.5 mmol) was combined with the reaction mixture. Ether (500 mL) and water (500 mL) were added and separated, organic phase was washed with brine (250 mL), dried over sodium sulfate, filtered and solvent removed in vacuo. Crude material was purified by flash column chromatography (20% EtOAc in petroleum ether) fractions were combined and desired isomer 3,4-Bis(benzyloxy)-2-methoxymethoxyphenol (2.95 g, 15%) was obtained as a brown oil. ¹H NMR (500 MHz, CDCl3) δ 7.45 −7.30 (m, 10H), 6.69 (d, J=9.0 Hz, 1H), 6.65 (d, J=9.0 Hz, 1H), 6.33 (dd, J=2.3, 1.1 Hz, 1H), 5.06 (s, 2H), 5.05 (s, 2H), 5.01 (d, J=1.0 Hz, 2H), 3.53 (s, 3H).

Synthesis of 1-(Allyloxy)-3,4-bis(benzyloxy)-2-methoxymethoxybenzene (9)

3,4-Bis(benzyloxy)-2-methoxymethoxyphenol (2.54 g, 6.96 mmol) were dissolved in DMF (20 mL), K₂CO₃ (1.38 g, 8.35 mmol) was added and stirred at room temperature overnight. Ether (100 mL) was added and washed with water (2×100 mL), dried over sodium sulfate, filtered and solvent removed in vacuo. 1-(Allyloxy)-3,4-bis(benzyloxy)-2-methoxymethoxybenzene (2.56 g, 90%) was obtained as a colourless oil. LCMS 407.2 [M+H]⁺, 98% Purity. ¹H NMR (500 MHz, CDCl₃) δ 7.51−7.30 (m, 10H), 6.68 (d, J=9.1 Hz, 1H), 6.61 (d, J=9.1 Hz, 1H), 6.09 (ddt, J=17.3, 10.6, 5.3 Hz, 1H), 5.43 (dq, J=17.2, 1.6 Hz, 1H), 5.29 (dq, J=10.5, 1.4 Hz, 1H), 5.20 (s, 2H), 5.10 (s, 2H), 5.06 (s, 2H), 4.54 (dt, J=5.3, 1.5 Hz, 2H), 3.60 (s, 3H).

Synthesis of 3-[3,4-Bis(benzyloxy)-2-methoxymethoxyphenoxy]-1,2-propanediol (10)

1-(Allyloxy)-3,4-bis(benzyloxy)-2-methoxymethoxybenzene (2.56 g, 6.31 mmol) was dissolved in THF (45 ml) and Water (5 mL), NMO (1.85 g, 15.76 mmol) was added followed by 4% OsO₄ (aq)(1.6 mL, 0.25 mmol), reaction was stirred at room temperature overnight. Sat. sodium bisulfite solution was added and extracted with DCM (3×60 mL), combined organics were washed with 1M HCI (60 mL) and brine (60 mL), dried over sodium sulfate and solvent removed in vacuo. 3-[3,4-Bis(benzyloxy)-2-methoxymethoxyphenoxy]-1,2-propanediol (2.78 g, quant.) LCMS 463.1 [M+Na]⁺ 85% Purity. ¹H NMR (500 MHz, CDCl₃) δ 7.50−7.29 (m, 10H), 6.69 (d, J=9.1 Hz, 1H), 6.64 (d, J=9.0 Hz, 1H), 5.16−5.11 (m, 2H), 5.08 (s, 2H), 5.07 (s, 2H), 4.11−3.97 (m, 3H), 3.85−3.79 (m, 1H), 3.77−3.72 (m, 1H), 3.57 (s, 3H). Alcohol O—H not visualised.

Synthesis of [3,4-Bis(benzyloxy)-2-methoxymethoxyphenoxy]acetaldehyde (11)

3-[3,4-Bis(benzyloxy)-2-methoxymethoxyphenoxy]-1,2-propanediol (2.78 g, 6.32 mmol) was dissolved in THF (50 mL) and water (25 mL), NaIO₄ (1.63 g, 7.59 mmol) was added. Reaction was stirred at room temperature for 2 hours. Water (50 mL) was added and extracted with EtOAc (3=75 mL), washed with brine, dried over sodium sulfate, filtered and solvent removed in vacuo. [3,4-Bis(benzyloxy)-2-methoxymethoxyphenoxy]acetaldehyde (2.2 g, 85%) was obtained as a brown oil. LCMS 409.1 [M+H]⁺ 75% Purity.

Synthesis of 2-[3,4-Bis(benzyloxy)-2-methoxymethoxyphenoxy]ethanol (12)

A solution of [3,4-Bis(benzyloxy)-2-methoxymethoxyphenoxy]acetaldehyde (2.2 g, 5.4 mmol) in IMS (22 mL) was cooled to 0 ° C., then NaBH4 was added and reaction warmed to RT after 15 minutes. After 2 hours, reaction was quenched with saturated NH4CI solution. Then extracted with EtOAc (3×50 mL), combined organics washed with Brine (100 mL), dried over sodium sulfate, filtered and solvent removed in vacuo. Crude material was purified by flash column chromatography (50% EtOAc in petroleum ether), fractions were combined and solvent removed in vacuo. 2-[3,4-Bis(benzyloxy)-2-methoxymethoxyphenoxy]ethanol (1.4 g, 63%) was obtained as a colourless oil. LCMS 428.2 [M+H₂O]⁺, 91% Purity.

Synthesis of 3,4-Bis(benzyloxy)-2-methoxymethoxy-1-[2-(o-nitrophenoxy)ethoxy]benzene (13)

3,4-Bis(benzyloxy)-2-methoxymethoxy-1-[2-(o-nitrophenoxy)ethoxy]benzene (1.4 g, 3.41 mmol) in THF (7.5 mL) was added to a suspension of 60% sodium hydride in mineral oil (137 mg, 3.41 mmol) in THF (7.5 mL) and stirred at room temperature for 30 minutes. 1-Fluoro-2-nitrobenzene (0.36 mL, 3.41 mmol) was added to the reaction mixture dropwise and then reaction mixture was heated to 60° C. After 1 hour, reaction mixture was cooled to room temperature, then poured into water and extracted with EtOAc (3×20 mL). Combined organics were washed with 1M NaOH, brine, dried over sodium sulfate, filtered and solvent removed in vacuo. Crude material was purified by flash column chromatography (2-60% EtOAc in petroleum ether), fractions combined and solvent removed in vacuo. 3,4- Bis(benzyloxy)-2-methoxymethoxy-1-[2-(o-nitrophenoxy)ethoxy]benzene (1.25 g, 69%) was obtained as a yellow oil. LCMS 549.3 [M+H₂O]⁺, 96% Purity ¹H NMR (500 MHz, CDCl₃) δ 7.84 (dd, J=8.1, 1.7 Hz, 1H), 7.56−7.27 (m, 11H), 7.16 (dd, J=8.5, 1.2 Hz, 1H), 7.06 (ddd, J=8.4, 7.4, 1.1 Hz, 1H), 6.68 (s, 2H), 5.14 (s, 2H), 5.07 (s, 2H), 5.05 (s, 2H), 4.45 (dd, J=5.8, 3.9 Hz, 2H), 4.37 (dd, J=5.8, 3.8 Hz, 2H), 3.53 (s, 3H).

Synthesis of o-{2-[3,4-Bis(benzyloxy)-2-methoxymethoxyphenoxy]ethoxy}aniline (14)

Zinc powder (620 mg, 9.41 mmol) was added to a suspension of 3,4-Bis(benzyloxy)-2-methoxymethoxy-1-[2-(o-nitrophenoxy)ethoxy]benzene (1.25 g, 2.35 mmol) and NH4CI (251 mg, 4.71 mg) in MeOH (20 mL) and water (4 mL). Reaction was stirred at room temperature for 1 hour. Reaction mixture was filtered through celite, washed through with MeOH. Solvent removed in vacuo. Residue was taken up in EtOAc (20 mL) washed with water (20 mL), brine (20 mL), dried over sodium sulfate, filtered and solvent removed in vacuo. To obtain o-{2-[3,4-Bis(benzyloxy)-2-methoxymethoxyphenoxy]ethoxy}aniline (622 mg, 69%) as a brown oil. ¹H NMR (500 MHz, CDCl3) δ 7.48−7.28 (m, 10H), 6.86−6.79 (m, 2H), 6.74−6.69 (m, 2H), 6.66 (d, J=3.4 Hz, 2H), 5.17 (s, 2H), 5.07 (s, 2H), 5.05 (s, 2H), 4.33 (m, 4H), 3.88 s, 2H), 3.54 (s, 3H).

Synthesis of N-(o-{2-[3,4-Bis(benzyloxy)-2-methoxymethoxyphenoxy]ethoxy}-phenyl)2,3-bis(benzyloxy)benzamide (15)

2,3-Bis(benzyloxy)benzoic acid (458 mg, 1.37 mmol) was dissolved in DMF (10 mL), EDC.HCI (286 mg, 1.49 mmol), NEt₃ (0.23 mL, 1.62 mmol), HOBt (202 mg, 1.49 mmol) were added, followed by o-{2-[3,4-Bis(benzyloxy)-2-methoxymethoxyphenoxy]ethoxy}aniline (622 mg, 1.24 mmol). Reaction was stirred at room temperature for 16 hours. Ether (100 mL) was added and washed with water (3×50 mL), dried over sodium sulfate, filtered and solvent removed in vacuo. Crude material was purified by flash column chromatography (10-50% EtOAc in petroleum ether), fractions were combined and solvent removed in vacuo. To obtain N-(o- {2-[3,4-Bis(benzyloxy)-2-methoxymethoxyphenoxy]ethoxy}phenyl)2,3-bis(benzyloxy)benzamide (520 mg, 51%) as a brown oil. LCMS 818.4 [M+H]⁺

Synthesis of N-(o-{2-[3,4-Bis(benzyloxy)-2-hydroxyphenoxy]ethoxy}phenyl)2,3-bis(benzyloxy)benzamide (16)

To a stirred solution of N-(o-{2-[3,4-Bis(benzyloxy)-2-methoxymethoxyphenoxy]ethoxy}phenyl) 2,3-bis(benzyloxy)benzamide (470 mg, 0.575 mmol) in DCM (8 mL) and MeOH (8 mL), 4M HCI in 1,4-dioxane (1.4 mL) was added and reaction mixture was stirred at room temperature for 2 hours. Solvent was removed in vacuo to obtain N-(o-{2-[3,4-Bis(benzyloxy)-2-hydroxyphenoxy]ethoxy}phenyl)2,3-bis(benzyloxy)benzamide (480 mg, 98%) as a red oil. LCMS 774.4 [M+H]⁺ 91% Purity.

Synthesis of (2S,3R,4S,5R,6R)-3,4,5-Triacetoxy-6-{[2,3-bis(benzyloxy)-6-(2-{o-[2,3-bis(benzyloxy)benzylamino]phenoxy}ethoxy)phenoxy]methyl}tetrahydro-2H-pyran-2-yl acetate (17)

To a stirred solution of N-(o-{2-[3,4-Bis(benzyloxy)-2-hydroxyphenoxy]-ethoxy}phenyl)2,3-bis(benzyloxy)benzamide (340 mg, 0.44 mmol) in anhydrous THF (8 mL) under nitrogen, was added (2S,3R,4S,5R,6R)-3,4,5-Triacetoxy-6-(hydroxymethy)tetrahydro-2H-pyran-2-yl acetate (155 mg, 0.439 mmol) then PPh3 (138 mg, 0.53 mmol). The solution was cooled to 0° C. and DIAD (104 μL, 0.527 mmol) was added dropwise. Reaction was stirred at room temperature for 16 hours and then concentrated in vacuo. The residue was purified by flash column chromatography (25-50% EtOAc in petroleum ether) fractions were combined, solvent removed in vacuo to obtain (2S,3R,4S,5R,6R)-3,4,5-Triacetoxy-6-{[2,3-bis(benzyloxy)-6-(2-{o-[2,3-bis-(benzyloxy)benzylamino]phenoxy}ethoxy)phenoxy]methyl} tetrahydro-2H-pyran-2-yl acetate (545 mg, quant.) as a colourless oil. UPLC 1104.1 [M+H]⁺ 74% Purity (91% purity at 254 nm).

Synthesis of N-{o[2-(2-{[(2R,3S,4S,5R,6R)-3,4,5,6-Tetrahydroxytetrahydro-2H-pyran-2-yl]methoxy}-3,4-bis(benzyloxy)phenoxy)ethoxy]phenyl}2,3-is(benzyloxy)benzamide (18)

(2S,3R,4S,5R,6R)-3,4,5-Triacetoxy-6-{[2,3-bis(benzyloxy)-6-(2-{o-[2,3-bis(benzyloxy)-benzylamino]phenoxy}ethoxy)phenoxy]methyl}tetrahydro-2H-pyran-2-yl acetate (545 mg, 0.494 mmol) was dissolved in MeOH (1 mL), 7M NH3 in MeOH (1 mL, 7.0 mmol) was added and stirred at room temperature. After 16 hours, solvent was removed in vacuo. N-{o[2-(2-{[(2R,3S,4S,5R,6R)-3,4,5,6-Tetrahydroxytetrahydro-2H-pyran-2-yl]methoxy}-3,4-bis(benzyloxy)-phenoxy)ethoxy]phenyl}2,3-bis(benzyloxy)benzamide (490 mg, 91%) was obtained as a gummy orange solid. UPLC 936.1 [M+H]⁺ (98% purity at 254 nm). Material used in the next step without further purification or characterisation.

Synthesis of N-{o[2-(2-{[(2R,3S,4S,5R,6R)-3,4,5,6-Tetrahydroxytetrahydro-2H-pyran-2-yl]methoxy}-3,4-dihydroxyphenoxy)ethoxy]phenyl}2,3-dihydroxybenzamide (19)

To a stirred solution of N-{o[2-(2-{[(2R,3S,4S,5R,6R)-3,4,5,6-Tetrahydroxytetrahydro-2H-pyran-2-yl]methoxy}-3,4-bis(benzyloxy)phenoxy)ethoxy]phenyl}2,3-bis(benzyl-oxy)benzamide (490 mg,0.523 mmol) in THF (10 mL) under an atmosphere of nitrogen, 10% Pd/C (56 mg, 0.052 mmol)was added. The reaction mixture was then stirred under an atmosphere of H2(g) for 26 hours. Reaction mixture was filtered through celite, washing with MeOH and then EtOAc. Filtrate then concentrated in vacuo. Crude material purified by reverse phase column chromatography (30-60% MeCN in water (0.1% Formic acid)) fractions were combined and solvent removed. Material was further purified by prep HPLC (30-35% MeCN in water (0.1% formic acid)), fractions combined and solvent removed. Purified material was further purified by reverse phase chromatography (25-35% MeCN in water (0.1% Formic acid)), fractions combined and solvent removed. To obtain N-{o-[2-(2-{[(2R,3S,4S,5R,6R)-3,4,5,6-Tetrahydroxytetrahydro-2H-pyran-2-yl]methoxy}-3,4-ihydroxyphenoxy)ethoxy]phenyl}2,3- dihydroxybenzamide (48 mg, 16%) as a white solid. UPLC 574.2 [M−H]⁻¹H NMR (500 MHz, d₆-DMSO) δ 8.38 (d, J=7.9 Hz, 1H), 7.46 (d, J=8.1 Hz, 1H), 7.19−7.17 (m, 1H), 7.10−7.07 (m, 1H), 7.00−6.97 (m, 2H), 6.79−6.70 (m, 1H), 6.46−6.38 (m, 2H), 4.95 (d, J=3.6 Hz, 1H), 4.42−4.31 (m, 4H), 4.16−3.86 (m, 4H), 3.19−3.11 (m, 2H), 2.97−2.94 (m, 1H). 8 ¹H unobserved; heteroatom protons. Material contains 23 w/w % diisopropylhydrazine.

### Example 7 In Vivo Testing of Compound of Formula 19 (MCF-013)

MCF-013 was formulated as a clear solution by dissolving in 10% final volume ethanol and then making up to full volume with saline at concentrations of 0.1, 0.3, 1 and 4 mg provided material/mL. This provided doses of 1, 3, 10 and 40 mg provided material/kg when administered intraperitoneally in 10 ml/kg dosing volumes.

Male CD-1 (ICR) mice were acclimatised to the procedure room. Groups of 3 mice were administered with vehicle (ethanol:saline) [10:90], or MCF-013 at 1, 3, 10 or 40 mg/kg intraperitoneally in 10 mL/kg dosing volumes. Doses were administered in an escalating dose order. The effect of MCF-013 on overt behavioural and physiological parameters was assessed using a modified Irwin procedure (Irwin S, 1968: A systematic, quantitative procedure for assessing the behavioural and physiologic state of the mouse. Animals were assessed over 5-minute periods at 6 predetermined time points after dosing (0.25, 0.5, 1, 1.5, 2, 4, 8 and 24 h). Each animal received only a single treatment. Immediately after the final assessment at 24 h, brains, liver, kidneys, heart, spleen, intestines and lungs from each mouse were observed for gross macroscopic changes compared to those of the control mice. These tissues were then excised and weighed and any with overt macroscopic abnormalities were to be placed in 10 volumes neutral buffered formalin solution (Sigma Cat no: HT501128).

### Results

Vehicle: One of three vehicle treated mice (No. 2) showed slight hunched posture at 0.25 and 0.5 h after dosing.

MCF-013 1 mg/kg: One of three mice (No. 6) showed slight hunched posture at 0.5 and 1h, and slight piloerection 0.5 h after dosing. No other behavioural or physiological changes were noted for this animal at other time point. The caecum of mouse 6 was noted to be slightly distended with air at gross macroscopic examination, but otherwise was normal in appearance and in the amount of digested food content. All other major organs for mouse 6 appeared normal as were organ weights MCF-013 3 mg/kg: One of three animals (mouse 9) showed slight hunched posture at 0.25 h after dosing. No other behavioural or physiological changes were noted at any other time point. Gross macroscopic examination of the major organs revealed no abnormalities and organ weights were normal.

MCF-013 10 mg/kg: no behavioural or physiological changes were noted. Gross macroscopic examination of the major organs revealed no abnormalities in any of the MCF-013 10 mg/kg dosed group and organ weights were normal.

MCF-013 40 mg/kg, no behavioural or physiological changes were noted for these animals at other time points. Gross macroscopic examination of the major organs revealed no abnormalities in any of the MCF-013 40 mg/kg dosed group and organweights were normal.

Conclusions MCF-013 given intraperitoneally at 1, 3, 10 and 40 mg provided material/kg appeared to be well tolerated over a 24 h period with no observations of adverse events that differed from those seen with 10 mL/kg of the ethanol:saline [10:90] vehicle. At the end of the study, no changes in the appearance of any major organs including stomach and food distribution were observed and all organ weights were normal. It is therefore concluded that MCF-013 was well tolerated in mice up to 40 mg provided material/kg and could be assessed in efficacy studies up to this dose level.

While certain embodiments of the invention have been illustrated and described, it will be clear that the invention is not limited to the embodiments described herein. Numerous modifications, changes, variations, substitutions, and equivalents will be apparent to those skilled in the art without departing from the spirit and scope of the present invention as described by the claims, which follow.

1. A conjugate comprising one or more compounds selected from (i) an anti-inflammatory compound; (ii) an antioxidant compound; (iii) an antibiotic compound; (iv) a metal chelating compound and at least
(v) a blood-brain-barrier targeting compound or
(vi) a solubility enhancer moiety,
wherein said (vi) solubility enhancer moiety and said compounds (i)-(v), when present, are covalently attached to each other via one or more linkers, the one or more linkers replacing a hydrogen or a heteroatom anywhere in said compounds (i)-(v) and said solubility enhancer moiety (vi), and wherein the one or more linkers are formed from one or more moieties each independently selected from a group consisting of unsubstituted or substituted C1-C10 alkylene, unsubstituted or substituted C2-C10 alkenylene, unsubstituted or substituted C2-C10 alkynylene, unsubstituted or substituted arylene, amide, ester, ether, thioether, amine, phosphonate, urea, sulfonamide, ethyleneglycol, polyethylene glycol, and carbamate,
and wherein the blood-brain-barrier targeting compound is a monosaccharide, and the solubility enhancer moiety is a phosphate group.

2. The conjugate according to claim 1, wherein the anti-inflammatory compound is indomethacin or naproxen.

3. The conjugate according to claim 1, wherein the antioxidant compound is resveratrol.

4. The conjugate according to claim 1, wherein the antibiotic compound is a polyketide antibiotic compound.

5. The composition according to claim 1, wherein the metal chelating compound is 4-{[(1,2-dimethyl-1,2-dihydropyridin-4-yl)meth-yl](ethyl)amino}butanoic acid or dihydroxybenzoic acid.

6. The conjugate according to claim 1, comprising a conjugate represented by either one of the following structures:

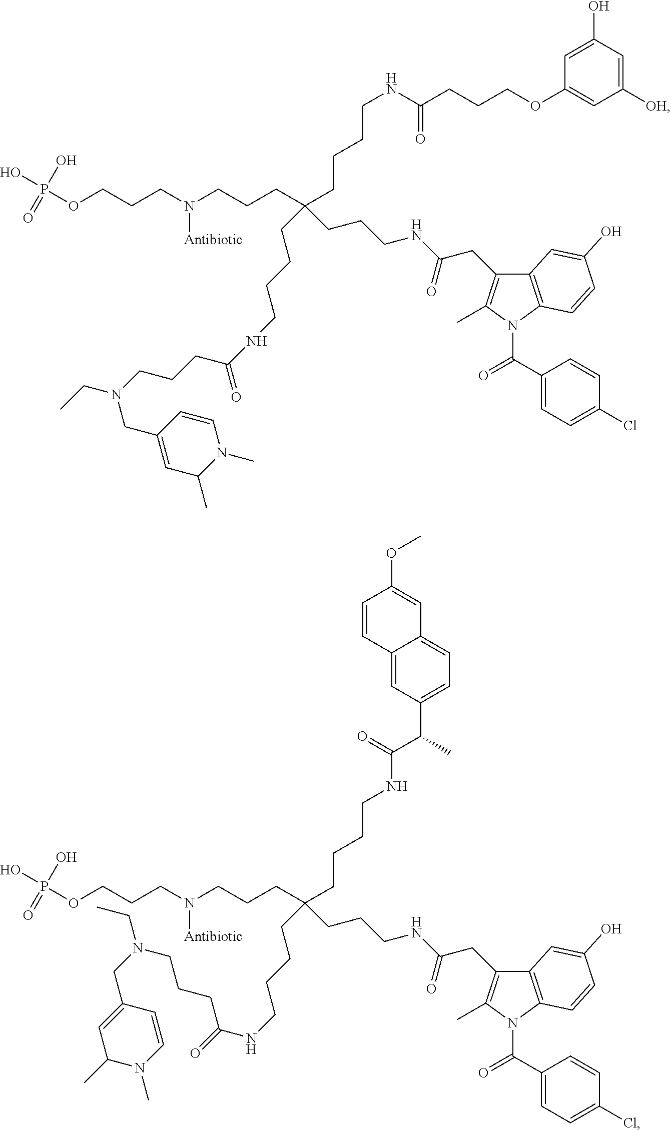

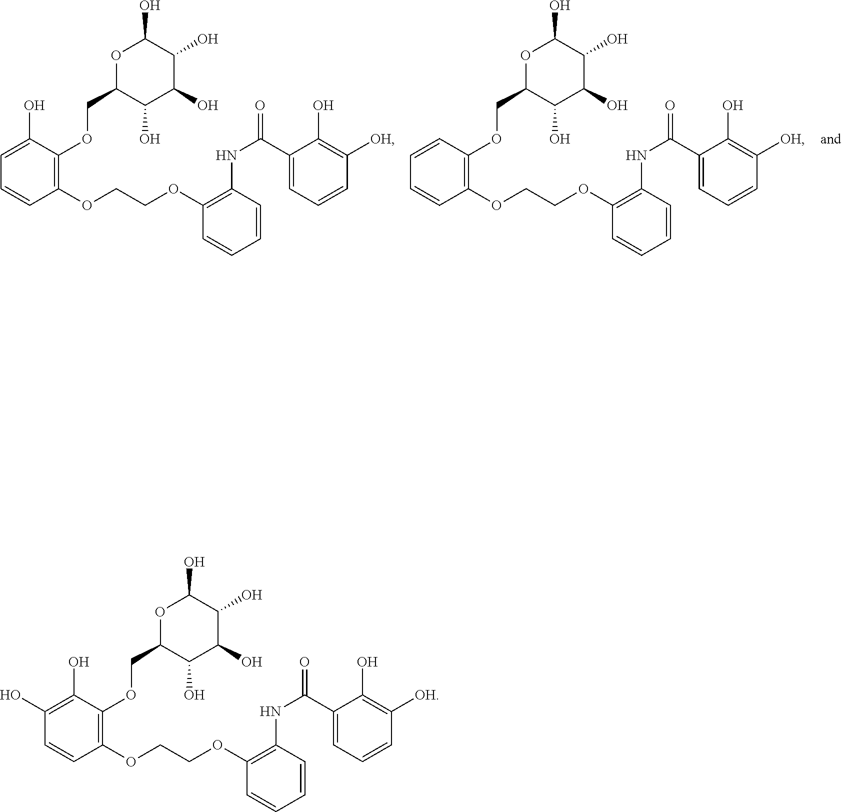

7. The conjugate according to claim 1, comprising a conjugate represented by either one of the following structures:

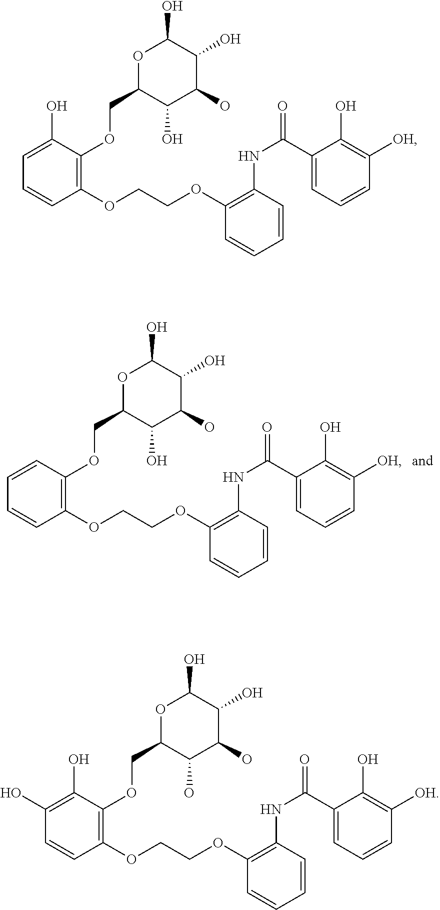

8. The conjugate according to claim 1. comprising a conjugate represented by the following structure:

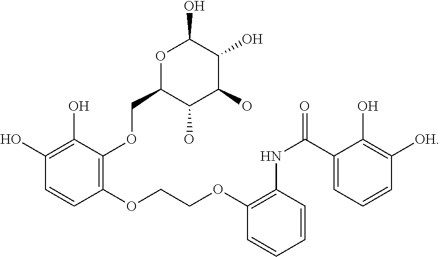

9. The conjugate according to claim 1 wherein at least one compound in said conjugate has at least two therapeutic functionalities selected from antioxidant, antibiotic, anti-inflammatory, metal chelation and blood-brain barrier targeting.

10. A composition comprising a conjugate according to claim 1 and one or more compounds selected from (i) an anti-inflammatory compound; (ii) an antioxidant compound; (iii) an antibiotic compound; (iv) a metal chelating compound and (v) a blood-brain barrier targeting compound.

11. A pharmaceutical composition comprising the conjugate according to claim 1 and a pharmaceutically acceptable carrier and/or excipient.

12. The pharmaceutical composition of claim 11, which is in the form of a capsule, tablet, granule, polymer, powder, solution or suspension and designed for administration by an oral sublingual, buccal, parenteral, intravenous, transdermal, inhalation, intranasal, vaginal, intramuscular or rectal mode.

13. A method for treating or preventing brain damage resulting from a brain injury, comprising administering an effective dose of the pharmaceutical composition of claim 11 to a patient in need thereof.

14. The method of claim 13, wherein the brain injury is traumatic brain injury (TBI).

15. The method of claim 13, wherein the brain damage is a secondary brain damage resulting from TBI.

16. The method of claim 13, wherein the brain injury is an acquired brain injury (ABI).

17. The method of claim 16, wherein the acquired brain injury results from stroke, brain tumour, brain tumour removal, infection, chemical or toxic poisoning, hypoxia, ischemia, encephalopathy, substance abuse, deceleration/blast injury or a combination thereof.

18. A method for treating or preventing a neurodegenerative disease or condition comprising administering an effective dose of the pharmaceutical composition of claim 11 to a patient in need thereof.

19. The method of claim 18, wherein the neurodegenerative disease or condition is selected from the group consisting of Alzheimer's Disease (AD), Multiple System Atrophy (MSA), Parkinsonism (Parkinson's syndrome, atypical Parkinson's, or secondary Parkinson's, including Parkinson's Disease), and Amyotrophic Lateral Sclerosis (ALS).

20. The conjugate according to claim 2, wherein the antioxidant compound is resveratrol.
